(19) United States
(12) Patent Application Publication
(10) Pub. No.: US 2026/0199234 A1
(43) Pub. Date: Jul. 16, 2026

(54) PHARMACEUTICAL COMPOSITIONS OF POLYMERIC MICELLAR CINACALCET, AND METHODS OF PREPARATION AND USE THEREOF

(71) Applicant: HANGZHOU SHIXI PHARMACEUTICAL TECHNOLOGY CO., LTD., Hangzhou, Zhejiang (CN)

(72) Inventors: Xiaoyan Sharon WANG, Hangzhou, Zhejiang (CN); Xichen ZHANG, Hangzhou, Zhejiang (CN)

(73) Assignee: HANGZHOU SHIXI PHARMACEUTICAL TECHNOLOGY CO., LTD., Hangzhou, Zhejiang (CN)

(21) Appl. No.: 18/996,702

(22) PCT Filed: Aug. 13, 2024
(86) PCT No.: PCT/CN2024/111860

§ 371 Date: Jan. 17, 2025

Related U.S. Application Data
(60) Provisional application No. 63/581,901, filed on Sep. 11, 2023.

(51) Int. Cl. A61K 9/10; A61K 31/137; A61K 47/26; A61K 47/34; A61K 47/40; A61P 5/20; A61P 5/22

(52) U.S. Cl. CPC A61K 9/10; A61K 31/137; A61K 47/26; A61K 47/34; A61K 47/40; A61P 5/20; A61P 5/22

(57) ABSTRACT

The present application provides pharmaceutical compositions of polymeric micellar cinacalcet suitable for intravenous or intraperitoneal administration, and methods of their preparation and use in treating various diseases and conditions (e.g., hyperparathyroidism and hypercalcemia).

### CROSS-REFERENCE TO RELATED APPLICATION

The present application claims the priority of the U.S. provisional patent application 63/581,901 filed with United States Patent and Trademark Office on Sep. 11, 2023, entitled “Pharmaceutical Compositions of Polymeric Micellar Cinacalcet, and Methods of Preparation and Use thereof”, which is incorporated herein by reference in its entirety.

### FIELD OF THE INVENTION

The present application generally relates to pharmaceuticals and therapeutic methods. More particularly, the present application provides pharmaceutical compositions of polymeric micellar cinacalcet, and methods of their preparation and use in treating various diseases and conditions (e.g., hyperparathyroidism and hypercalcemia).

### BACKGROUND OF THE INVENTION

Cinacalcet is a calcimimetic agent that increases the sensitivity of the calcium-sensing receptor to activation by extracellular calcium. For example, cinacalcet is used to reduce parathyroid hormone (PTH), serum calcium, serum phosphorus, and the calcium-phosphorus product in patients with chronic kidney disease and secondary hyperparathyroidism who are receiving dialysis, and reduces elevated serum calcium associated with primary hyperparathyroidism and parathyroid carcinoma.

Sold under the brand names Sensipar® and Mimpara® among others, existing commercial cinacalcet products are formulated as solid tablets and are administered orally. Studies have shown that solid tablets of cinacalcet are poorly absorbed. When taken orally, cinacalcet is dissolved in stomach where pH is low, but it can precipitate in the lower GI track where pH is higher. As such, the dissolution and bioavailability of cinacalcet tablets are not optimal, with low bioavailability and high variabilities in pharmacokinetic profiles. The absolute bioavailability of cinacalcet in fasted subjects has been estimated at 20-25%. (EMEA Mimpara Summary of Product Characteristics-Section 5.2 Pharmacokinetic Properties, https://www.ema.europa.eu/en/documents/product-information/mimpara-epar-product-information_en.pdf). The labeling information issued by the FDA and EMEA for Sensipar® and Mimpara® include a recommendation that cinacalcet be taken with food or shortly after a meal. If a patient fails to take the medication exactly as indicated, sub-therapeutic levels of cinacalcet can occur. Patient compliance to oral cinacalcet has remained a significant challenge.

Various attempts have been made to improve patient compliance with efforts on intravenous formulations as they can be conveniently administered during dialysis. Cinacalcet's poor water solubility and blood compatibility have hindered the development of intravenous formulation. One example of such effort was to dissolve cinacalcet in oil, followed by emulsification in water (Gore et al, 2014, U.S. Pat. No. 8,779,004). However, the oil-in-water emulsion formulation was less stable, with difficulty to control particle size distributions. In addition, the emulation formulation had limited capability of achieving long lasting circulation in blood.

To date, suitable intravenous cinacalcet formulations have not been successfully developed. There remains an urgent unmet need for suitable intravenous cinacalcet formulations to help improve patient compliance and treatment outcome.

### SUMMARY OF THE INVENTION

The present application is based in part on the discovery of polymeric micellar formulations of cinacalcet suitable for intravenous or intraperitoneal administration, for example, during dialysis. The polymeric micellar cinacalcet formulations disclosed herein exhibit stability upon dilution, blood compatibility, improved safety by reducing injection site reactions, efficacious in reducing PTH level, and can be easily and economically manufactured.

More particularly, pharmaceutical formulations of cinacalcet and methods of preparation and use thereof are provided herein. The disclosed pharmaceutical formulations and therapeutic methods are suitable for delivering cinacalcet to a patient suffering from a disease in need of treatment with cinacalcet. The pharmaceutical formulations of the present application comprise cinacalcet solubilized by amphiphilic block copolymers, as well as other excipients, such as one or more of buffering agents, cryo- and lyophilization protectants or bulking agents, and surfactants. The polymeric micelle cinacalcet compositions of the present application can be formulated into a stable lyophilized form for long term storage. Upon reconstitution, colloidal polymeric micelles are formed suitable for injection. It was discovered that cyclodextrins and their derivatives effectively protect the micelles made of water-insoluble polymers, preventing aggregation of the micelles.

The polymeric micelle formulations are readily prepared by combining cinacalcet/block copolymer organic solution with aqueous buffer solution. The solvent may be optionally removed by evaporation, and other excipients may be added as needed. Alternatively, the polymeric micelle formulation may be prepared by dissolving cinacalcet and block copolymer in an organic solvent, removing solvent by evaporation under vacuum and modest heat to form a solid matrix, and reconstituting with an aqueous buffer optionally containing lyo-protectant or bulking agent. The micelle solution can be lyophilized and reconstituted before use. The micellar formulations are stable upon frozen and/or lyophilization, and stable during long-term storage.

The formulations are blood compatible, therefore circumventing phlebitis and reducing pain upon intravenous administration. The micelle formulation can prolong cinacalcet circulation in blood. The formulation is suitable for parenteral administration, e.g., intravenously or intraperitoneally, to patient during dialysis to increase patient compliance over oral cinacalcet tablets. The pharmaceutical formulations can be used for any disease that is sensitive to the treatment with cinacalcet, such as controlling PTH level in kidney dialysis patients.

In one aspect, the present application generally relates to the pharmaceutical composition of cinacalcet, comprising micelles comprising cinacalcet, or a pharmaceutically acceptable salt thereof, and at least one amphiphilic block copolymer.

In another aspect, the present application generally relates to the unit dosage form comprising the pharmaceutical composition.

In yet another aspect, the present application generally relates to a method for controlling PTH levels, comprising administering to a subject in need thereof a therapeutically effective amount of the pharmaceutical composition or the unit dosage form disclosed herein.

In yet another aspect, the present application generally relates to a method for regulating calcium and phosphorus in blood, comprising administering to a subject in need thereof a therapeutically effective amount of the pharmaceutical composition or the unit dosage form disclosed herein.

In yet another aspect, the present application generally relates to a method for treating primary hyperparathyroidism, comprising administering to a subject in need thereof a therapeutically effective amount of the pharmaceutical composition or the unit dosage form disclosed herein.

In yet another aspect, the present application generally relates to a method for treating secondary hyperparathyroidism, comprising administering to a subject in need thereof a therapeutically effective amount of the pharmaceutical composition or the unit dosage form disclosed herein.

In yet another aspect, the present application generally relates to a method for treating parathyroid carcinoma, comprising administering to a subject in need thereof a therapeutically effective amount of the pharmaceutical composition or the unit dosage form disclosed herein.

In yet another aspect, the present application generally relates to a method for preparing a composition of polymeric micellar drug disclosed herein, comprising: dissolving at least one amphiphilic block copolymer and a hydrophobic drug, or a pharmaceutically acceptable salt thereof, in an organic solvent, forming a first mixture; adding the first mixture to water or an aqueous solution with stirring; optionally removing the organic solvent, resulting in clear to translucent colloidal micelles; and optionally adding one or more pharmaceutically acceptable excipients, carriers or diluents. The micellar formulations can be further lyophilized to remove water and solvent, if present. The micellar formulation can be stored frozen or in refrigerator upon lyophilized, and stable during long-term storage.

In yet another aspect, the present application generally relates to another method for preparing a composition of polymeric micellar cinacalcet disclosed herein, comprising: dissolving at least one amphiphilic block copolymer and a hydrophobic drug, or a pharmaceutically acceptable salt thereof, in an organic solvent, forming a first mixture; removing the organic solvent by evaporation, and forming a matrix; adding water or an aqueous solution to the matrix, resulting in clear to translucent colloidal micelles; and optionally adding one or more pharmaceutically acceptable excipients, carriers or diluents. The micellar formulations can be further lyophilized to remove water. The micellar formulation can be stored frozen or in refrigerator upon lyophilized, and stable during long-term storage.

In yet another aspect, the present application generally relates to use of the pharmaceutical composition disclosed herein for controlling PTH levels.

In yet another aspect, the present application generally relates to use of the pharmaceutical composition disclosed herein for regulating calcium and phosphorus in blood.

In yet another aspect, the present application generally relates to use of the pharmaceutical composition disclosed herein for treating primary hyperparathyroidism.

In yet another aspect, the present application generally relates to use of the pharmaceutical composition disclosed herein for treating secondary hyperparathyroidism.

In yet another aspect, the present application generally relates to use of the pharmaceutical composition disclosed herein for treating hypercalcemia.

In yet another aspect, the present application generally relates to the pharmaceutical composition disclosed herein for use in controlling parathyroid hormone (PTH) levels.

In yet another aspect, the present application generally relates to the pharmaceutical composition disclosed herein for use in regulating calcium and phosphorus in blood.

In yet another aspect, the present application generally relates to the pharmaceutical composition disclosed herein for use in treating primary hyperparathyroidism.

In yet another aspect, the present application generally relates to the pharmaceutical composition disclosed herein for use in treating secondary hyperparathyroidism.

In yet another aspect, the present application generally relates to the pharmaceutical composition disclosed herein for use in treating parathyroid carcinoma.

In yet another aspect, the present application generally relates to use of cinacalcet, or a pharmaceutically acceptable salt thereof, and at least one amphiphilic block copolymer in the manufacture of a medicament.

Notably, the compositions of the present application are easy to manufacture, stable upon freezing and lyophilization, and stable in long term storage. The pharmaceutical compositions are blood compatible and reduce phlebitis and pain upon intravenous administration. The selected amphiphilic copolymers can provide sustained release of cinacalcet, and can increase circulation time in blood, therefore reducing dosing frequency. The pharmaceutical formulations can be conveniently administered intravenously during dialysis resulting in increased patient compliance, increased bioavailability and reduced variabilities in pharmacokinetic parameters, eliminating “food effect” as compared to oral cinacalcet tablet formulations.

### BRIEF DESCRIPTION OF THE DRAWINGS

FIG. 1 shows a flow chart illustrating an exemplary manufacturing process for preparing the formulations of the present application.

FIG. 2 shows an exemplary polymer molecular weight distribution of PDLLA-MePEG 2-2 (lot NB009-029).

FIG. 3 shows an exemplary particle size distribution of cinacalcet polymer micelles.

FIG. 4 shows certain exemplary data on prolonged in vitro release of cinacalcet from the polymeric micellar formulations of the present application, through a 12-14 kD dialysis membrane, into a 20 mM PBS buffer of pH 7.4 with 0.1% PS80. The temperature was maintained at 37° C.

FIGS. 5a-5b show certain exemplary data on improved blood compatibility of the formulations of the present application.

FIG. 6 shows certain exemplary data on physical stability of the liquid polymeric micellar cinacalcet formulations (CO, CP, CR, CT, DO, DP, DR, DS, DU, DV) stored frozen at −20° C. Turbidity of thawed and 15× diluted samples vs storage time is shown.

FIG. 7 shows certain exemplary data on physical stability of a lyophilized polymeric micellar cinacalcet formulation (DX) stored at 2-8° C. Turbidity of reconstituted and 15× diluted samples vs storage time is shown.

FIG. 8 shows reduction in PTH level after iv dosing of cinacalcet in adult male SD rats, on day 1, 3, 5, 8 (Rat Study 1).

FIGS. 9a-9f show plasma drug concentration profiles after iv dosing of cinacalcet in male SD rats (Rat Study 2).

FIG. 10 shows rat body weights, where the cinacalcet buffer solution group has different and lower weight gain (Rat Study 3).

FIG. 11 shows rat tail diameter close to the body, indicating reduction of swelling and inflammation from the cinacalcet polymer micelle formulation (Rat Study 4).

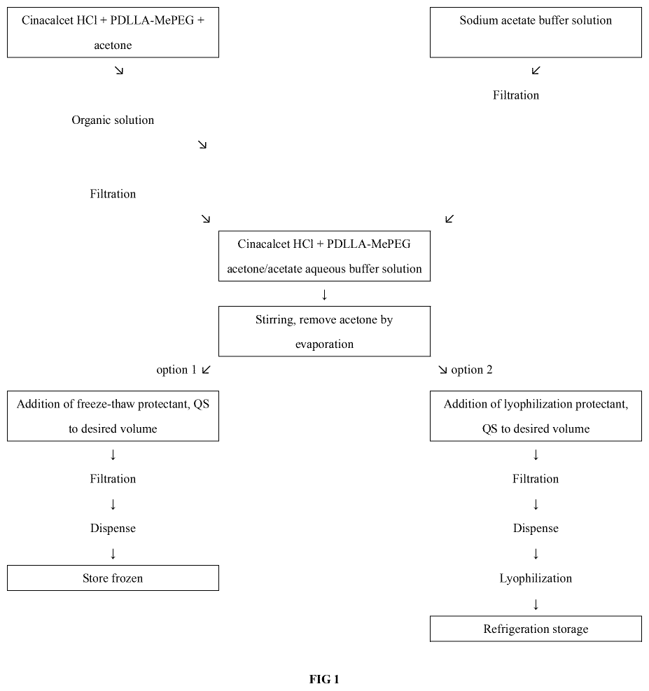

### Definitions

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present disclosure, the preferred methods and materials are now described. Methods recited herein may be carried out in any order that is logically possible, in addition to a particular order disclosed.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the present application. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

In this specification and the appended claims, the singular forms “a,” “an,” and “the” include plural reference, unless the context clearly dictates otherwise.

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure

As used herein, “at least” a specific value is understood to be that value and all values greater than that value.

The term “comprising”, when used to define compositions and methods, is intended to mean that the compositions and methods include the recited elements, but do not exclude other elements. The term “consisting essentially of”, when used to define compositions and methods, shall mean that the compositions and methods include the recited elements and exclude other elements of any essential significance to the compositions and methods. For example, “consisting essentially of” refers to administration of the pharmacologically active agents expressly recited and excludes pharmacologically active agents not expressly recited. The term consisting essentially of does not exclude pharmacologically inactive or inert agents, e.g., pharmaceutically acceptable excipients, carriers or diluents. The term “consisting of”, when used to define compositions and methods, shall mean excluding trace elements of other ingredients and substantial method steps. Embodiments defined by each of these transition terms are within the scope of this invention.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

As used herein, the term “administration” of a disclosed compound encompasses the delivery to a subject of a compound as described herein, or a salt or other pharmaceutically acceptable form thereof, using any suitable formulation or route of administration, as discussed herein.

The terms “disease”, “disorder” and “condition” are used interchangeably unless indicated otherwise.

As used herein, the term “pharmaceutically acceptable excipient, carrier, or diluent” generally refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient.

As used herein, a “pharmaceutically acceptable form” of a disclosed compound includes, but is not limited to, pharmaceutically acceptable salts, esters, hydrates, solvates, isomers, prodrugs, and isotopically labeled derivatives of disclosed compounds. In one embodiment, a “pharmaceutically acceptable form” includes, but is not limited to, pharmaceutically acceptable salts, esters, isomers, prodrugs and isotopically labeled derivatives of disclosed compounds.

In one or more embodiments, the pharmaceutically acceptable form is a pharmaceutically acceptable salt. “Pharmaceutically acceptable salts” of the compounds described herein include those derived from said compounds when mixed with inorganic or organic acids or bases. In some embodiments, the salts can be prepared in situ during the final isolation and purification of the compounds. In other embodiments, the salts can be prepared from the free form of the compounds in a separate synthetic step. The preparation of the pharmaceutically acceptable salts described above, and other typical pharmaceutically acceptable salts is more fully described by Berg et al., “Pharmaceutical Salts,” J. Pharm. Sci., 1977:66:1-19, incorporated here by reference in its entirety. The pharmaceutically acceptable salts of the compounds described herein are those that may be used in medicine. Salts that are not pharmaceutically acceptable may, however, be useful in the preparation of the compounds described herein of their pharmaceutically acceptable salts.

The compounds or pharmaceutically acceptable salts thereof as described herein, may contain an asymmetric carbon atom, for example, as the result of deuterium substitution or otherwise. As such, compounds of this invention can exist as either individual enantiomers, or mixtures of the two enantiomers. Accordingly, a compound of the present invention may exist as either a racemic mixture or a scalemic mixture, or as individual respective stereoisomers that are substantially free from another possible stereoisomer. The term “substantially free of other stereoisomers” as used herein means less than 25% of other stereoisomers, preferably less than 10% of other stereoisomers, more preferably less than 5% of other stereoisomers and most preferably less than 2% of other stereoisomers are present. Methods of obtaining or synthesizing an individual enantiomer for a given compound are known in the art and may be applied as practicable to final compounds or to starting material or intermediates.

In one or more embodiments, the pharmaceutically acceptable form is a “solvate” (e.g., a hydrate). As used herein, the term “solvate” refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate”. Pharmaceutically acceptable solvates and hydrates are complexes that, for example, can include 1 to about 100, or 1 to about 10, or 1 to about 2, about 3 or about 4, solvent or water molecules. It will be understood that the term “compound” as used herein encompasses the compound and solvates of the compound, as well as mixtures thereof.

As used herein, the term “stable” when used to describe a compound or pharmaceutical composition refers to the compound or pharmaceutical composition possessing chemical and physical stabilities sufficient to allow for their manufacture and to maintain the integrity of such compound or pharmaceutical composition for a sufficient period of time to be useful for the intended purposes detailed herein (e.g., formulation into therapeutic products, treatment of a disease or condition responsive to the therapeutic agents).

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject in need of treatment.

As used herein, the term “therapeutic effect” refers to a therapeutic benefit and/or a prophylactic benefit as described herein. A prophylactic effect includes delaying or eliminating the appearance of a disease or condition, delaying or eliminating the onset of symptoms of a disease or condition, slowing, halting, or reversing the progression of a disease or condition, or any combination thereof.

As used herein, the term “therapeutically effective amount” refers to that amount of a compound or pharmaceutical composition described herein that is sufficient to effect the intended application including, but not limited to, disease treatment, as illustrated below. The therapeutically effective amount can vary depending upon the intended application, or the subject and disease condition being treated, e.g., the desired biological endpoint, the pharmacokinetics of the compound, the disease being treated, the mode of administration, and the weight and age of the patient, which can readily be determined by one of ordinary skill in the art. The specific dose will vary depending on, for example, the particular compounds chosen, the species of subject and their age/existing health conditions or risk for health conditions, the dosing regimen to be followed, the severity of the disease, whether it is administered in combination with other agents, timing of administration, the tissue to which it is administered, and the physical delivery system in which it is carried.

As used herein, the term “treatment” or “treating” a disease or disorder refers to a method of reducing, delaying or ameliorating such a condition before or after it has occurred. Treatment may be directed at one or more effects or symptoms of a disease and/or the underlying pathology. Treatment is aimed to obtain beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For prophylactic benefit, the pharmaceutical compounds and/or compositions can be administered to a patient at risk of developing a particular disease, or to a patient reporting one or more of the physiological symptoms of a disease, even though a diagnosis of this disease may not have been made. The treatment can be any reduction and can be, but is not limited to, the complete ablation of the disease or the symptoms of the disease. As compared with an equivalent untreated control, such reduction or degree of prevention is at least 5%, 10%, 20%, 40%, 50%, 60%, 80%, 90%, 95%, or 100% as measured by any standard technique.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

### DETAILED DESCRIPTION OF THE INVENTION

The detailed descriptions, particular embodiments and examples are provided by way of illustration and not by way of limitation. Those skilled in the art will readily recognize a variety of noncritical parameters that could be changed or modified to yield essentially similar results. All publications, patents, and patent applications cited herein, including citations therein, are hereby incorporated by reference in their entirety for all purposes.

The present application provides stable polymeric micellar formulations of cinacalcet suitable for intravenous or intraperitoneal administration to a patient suffering from a disease in need of treatment with cinacalcet. The pharmaceutical formulation of the present application comprises cinacalcet solubilized by amphiphilic block copolymers, as well as other excipients, such as one or more of buffering agents, cryo- and lyophilization protectants or bulking agents, and surfactants.

The polymeric micelle formulation of the present application is blood compatible, can circumvent phlebitis and reduce pain upon intravenous administration. The formulation of the present application can prolong cinacalcet circulation in blood and is suitable for parenteral administration, e.g., intravenously or intraperitoneally, to patient during dialysis to increase patient compliance over oral cinacalcet tablets. In addition, the formulation of the present application exhibits stability upon dilution and during long-term storage, blood compatibility with prolonged circulation in blood, not causing phlebitis and pain, and having adequate pharmacokinetic profile (e.g., long lasting and low variabilities), and can be easily and economically manufactured as well.

The polymeric micelle formulations of the present application are readily prepared by one of the following methods: 1) combining cinacalcet/block copolymer organic solution with aqueous buffer solution, followed by optionally solvent evaporation and addition of other excipients as needed; or 2) combining cinacalcet/block copolymer organic solution, evaporating the solvent, followed by dissolving the drug/block copolymer matrix in an aqueous buffer solution with addition of other excipients as needed. The micellar formulations of the present application are stable upon frozen and/or lyophilization, and stable during long-term storage.

The pharmaceutical formulation of the present application can be used to treat a disease that is sensitive to the treatment with cinacalcet, such as controlling PTH level in kidney dialysis patients. More generally, the cinacalcet formulation disclosed herein can be used to treat hyperparathyroidism (elevated PTH levels) and the symptoms thereof. Hyperparathyroidism is overactivity of the parathyroid glands resulting in excess production of PTH. PTH regulates calcium and phosphate levels and helps to maintain these levels. Overactivity of one or more of the parathyroid glands causes high calcium levels (hypercalcemia) and low levels of phosphate in the blood. Hyperparathyroidism may be a consequence of parathyroid tumors and chronic renal failure.

Cinacalcet has the chemical structure shown below, which has a chiral center with an R-absolute configuration. The R-enantiomer is the more potent enantiomer and has been shown to be responsible for pharmacodynamic activity (https://www.pi.amgen.com/~/media/amgen/repositorysites/pi-amgen-com/sensipar/sensipar_pi_hcp_english.pdf).

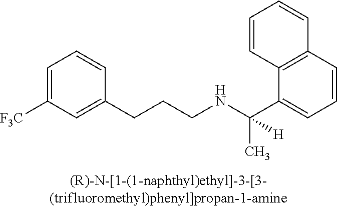 [SMILES: C[C@@H](NCCCc1cccc(C(F)(F)F)c1)c1cccc2ccccc12]

Polymeric micellar formulation of the present application utilizes amphiphilic block copolymers, often diblock copolymers (i.e., one block is hydrophobic polymer, another block is hydrophilic polymer). Comparing to traditional low molecular weight surfactants, the hydrophobic block of the diblock copolymers can have higher molecular weight, more hydrophobic, and more rigid (e.g., higher glass transition temperature). Hence, the critical micelle concentration of polymeric micelles of the present application is much lower and the drug loading capacity can be much higher. Other amphiphilic block copolymers, such as triblock copolymers (e.g., two blocks are hydrophobic polymers, another block is hydrophilic polymer), can also be utilized and have similar characteristics.

Block copolymers of polyethylene glycol or methoxypolyethylene glycol and biodegradable polyesters of polylactide, polyglycolide, poly(ε-caprolactone), and their copolymers can form micelles. Long lasting and sustained release effect can be obtained using higher molecular weight polylactide or other polyesters, as longer polyester chain will increase hydrophobic association and resist dissociation in presence of blood components. However, the very insoluble nature of the longer polymer chain introduces challenges in formulating and manufacturing. To manufacture micelle formulation, both the copolymer and drug are often dissolved in an organic solvent, such as acetonitrile and acetone, and then mixed with an aqueous solution. After solvent evaporation, micelles or even nanoparticle (in the case of using even longer polyester chain) can be formed, resulting in drug loaded polymeric colloid in water. Drug crystallization often occurs during solvent evaporation, hence requires delicate process which often renders it non-economical. Water-insoluble dichloromethane (DCM) is also used, to alleviate drug precipitation in the case of water miscible acetone and acetonitrile. However, DCM is more toxic and less preferred. Due to hydrolysis of polyester in presence of water, the formulation needs to be stored either as frozen liquid or lyophilized, for long term stability. Unfortunately, these micelles are very susceptible to aggregation and phase separation upon freezing and lyophilization. Therefore, freezing and lyophilization need to be carefully studied for cryo- and lyo-protectants and process conditions (Abdelwahed W et al 2006, Adv. Drug Delivery Reviews, 58, 1688-1713; Fonte P et al, 2016, J Contr. Rel., 225, 75-86).

In one aspect, the present application generally relates to a pharmaceutical composition of cinacalcet, comprising micelles comprising cinacalcet, or a pharmaceutically acceptable salt thereof, and at least one amphiphilic block copolymer. In one or more embodiments, the amphiphilic block copolymer is a diblock copolymer. In one or more embodiments, the amphiphilic block copolymer is biodegradable polyester-block-methoxy polyethylene glycol (MePEG).

In one or more embodiments, the amphiphilic block copolymer is a triblock copolymer. In one or more embodiments, the amphiphilic block copolymer is biodegradable polyester-block-polyethylene glycol (PEG)-block-polyester.

In one or more embodiments, the polyester block is selected from poly(D,L-lactide), poly(L-lactide), poly(D-lactide), polyglycolide, poly(ε-caprolactone), and copolymers and mixtures thereof. In one or more embodiments, the polyester block is poly(D,L-lactide) (PDLLA). In one or more embodiments, the molecular weight (Mₙ) of PDLLA is within the range from about 750 to about 50,000 (e.g., from about 1,000 to about 30,000, from about 1,300 to about 30,000, from about 1,000 to about 20,000, from about 1,300 to about 20,000).

In one or more embodiments, the molecular weight of MePEG is within the range from about 750 to about 20,000 (e.g., from about 1,200 to about 10,000, from about 1,300 to about 6,000, from about 1,900 to about 5,000).

In one or more embodiments, the molecular weight of PEG is within the range from about 200 to about 20,000 (e.g., from about 750 to about 20,000, from about 1,200 to about 10,000, from about 1,300 to about 6,000).

The pharmaceutical compositions of the present application may use any suitable forms of cinacalcet, e.g., cinacalcet HCl. Acceptable cinacalcet salts can be derived from inorganic or organic acids, including, but not limited to: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxy-ethanesulfonate, lactate, maleate, mandelate, methansulfonate, nicotinate, 2-naphthalenesulfonate, oxalate, palmoate, pectinate, persulfate, 2-phenylpropionate, picrate, pivalate, propionate, salicylate, succinate, sulfate, tartrate, thiocyanate, tosylate, mesylate, and undecanoate.

In one or more embodiments, the cinacalcet HCl loading in the amphiphilic block copolymer is from about 1% to about 70%, e.g., from about 5% to about 20%, from about 8% to about 12%.

In one or more embodiments, the pharmaceutical composition further comprises one or more pharmaceutically acceptable excipients, carriers or diluents.

In one or more embodiments, the one or more pharmaceutically acceptable excipients, carriers or diluents are selected from buffering agents, osmolality agents, cryopreservation protectants, lyophilization protectants or bulking agents, surfactants, and solvents.

In one or more embodiments, the pharmaceutical composition comprises one or more cryopreservation and lyophilization protectants or bulking agents selected from mannitol, sucrose, trehalose, cyclodextrins and derivatives thereof.

In or more embodiments, the pharmaceutical composition comprises α-CD, β-CD, γ-CD hydroxypropyl-β-cyclodextrin (HPBCD), and/or sulfobutylether-β-cyclodextrin (SBEβCD).

In one or more embodiments, the pharmaceutical composition comprises HPBCD at a concentration within the range from about 3% to about 10%, e.g., from about 4% to about 8%, from about 5% to about 7% (wt/v %).

It is noted that where a concentration is defined by weight/volume percentage (wt/v %), such concentration refers to an embodiment of the present application wherein the composition is a liquid (e.g., aqueous) composition with one or more solvents. In all other situations, a concentration (%) refers to the weight percentage of a component in a composition.

In one or more embodiments, the pharmaceutical composition comprises a surfactant selected from polysorbates and poloxamers. In one or more embodiments, the polysorbates and poloxamers are selected from polysorbate 20, polysorbate 80, and Pluronic® 68. In one or more embodiments, the pharmaceutical composition comprises polysorbate 80 at a concentration within the range from about 0.01% to about 0.5%, e.g., from about 0.05% to about 0.5%, from about 0.1% to about 0.5%, from about 0.01% to about 0.1% (wt/v %).

In one or more embodiments, the pharmaceutical composition comprises a buffering agent. In one or more embodiments, the buffering agent is selected from acetate, citrate, and phosphate. In one or more embodiments, the pharmaceutical composition comprises acetic acid/sodium acetate or citric acid/sodium citrate with a pH in the range from about 4.0 to about 7.9 (e.g., from about 4.0 to about 6.0, from about 6.0 to about 7.0, from about 7.0 to about 7.9).

In one or more embodiments, the pharmaceutical composition is an aqueous colloid. In one or more embodiments, the pharmaceutical composition is clear to translucent colloid. In one or more embodiments, the average particle size of polymeric micelles the pharmaceutical composition in less than about 200 nm (e.g., less than about 175 nm, less than about 150 nm, less than about 50 nm).

In one or more embodiments, the pharmaceutical composition is in a solution form in organic solvent. In one or more embodiments, the pharmaceutical composition is in an aqueous liquid form that can be stored frozen. In one or more embodiments, the pharmaceutical composition is in a matrix form. In one or more embodiments, the pharmaceutical composition is in a lyophilized form.

In one or more embodiments, the pharmaceutical composition is suitable for reconstitution and administration of cinacalcet.

In another aspect, the present application generally relates to a unit dosage form comprising the pharmaceutical composition.

In one or more embodiments, the unit dosage form is suitable for intravenous administration. In one or more embodiments, the unit dosage form is suitable for intraperitoneal administration.

In yet another aspect, the present application generally relates to a method for controlling PTH levels, comprising administering to a subject in need thereof a therapeutically effective amount of the pharmaceutical composition or the unit dosage form disclosed herein.

In yet another aspect, the present application generally relates to a method for regulating calcium and phosphorus in blood, comprising administering to a subject in need thereof a therapeutically effective amount of the pharmaceutical composition or the unit dosage form disclosed herein.

In yet another aspect, the present application generally relates to a method for treating primary hyperparathyroidism, comprising administering to a subject in need thereof a therapeutically effective amount of the pharmaceutical composition or the unit dosage form disclosed herein.

In yet another aspect, the present application generally relates to a method for treating secondary hyperparathyroidism, comprising administering to a subject in need thereof a therapeutically effective amount of the pharmaceutical composition or the unit dosage form disclosed herein.

In yet another aspect, the present application generally relates to a method for treating parathyroid carcinoma, comprising administering to a subject in need thereof a therapeutically effective amount of the pharmaceutical composition or a unit dosage form disclosed herein.

In one or more embodiments of these methods, the administration is intravenous administration. In one or more embodiments of these methods, the administration is intraperitoneal administration. In one or more embodiments of these methods, the administration is performed immediately prior to, during or immediately after the subject's dialysis procedure. In one or more embodiments of these methods, the administration is performed during the subject's dialysis procedure.

In one or more embodiments of these methods, the administration of the pharmaceutical composition provides sustained release of cinacalcet.

In one or more embodiments of these methods, the administration of the pharmaceutical composition causes no phlebitis.

In yet another aspect, the present application generally relates to a method for preparing the composition of polymeric micellar cinacalcet disclosed herein, comprising: dissolving at least one amphiphilic block copolymer and cinacalcet, or a pharmaceutically acceptable salt thereof, in an organic solvent, forming a first mixture; forming cinacalcet polymer micelles in one of A, B, or C approach; A: adding the first mixture to water or an aqueous solution with stirring, resulting in clear to translucent colloidal cinacalcet micelles; B: adding the first mixture to water or an aqueous solution with stirring; removing the organic solvent, resulting in clear to translucent colloidal cinacalcet micelles; C: removing the organic solvent followed by adding an aqueous buffer, resulting in clear to translucent colloidal cinacalcet micelles; and then optionally adding one or more pharmaceutically acceptable excipients, carriers or diluents. The aqueous micellar solutions can be stored frozen or lyophilized for long term storage.

In one or more embodiments of the preparation method, the organic solvent is selected from ethanol, acetone, acetonitrile, dichloromethane (DCM), isopropanol, ethyl acetate, tert-butanol, and methanol.

In one or more embodiments of the preparation method, removing the organic solvent is via evaporation.

In one or more embodiments of the preparation method, the amphiphilic block copolymer is a diblock copolymer.

In one or more embodiments of the preparation method, the amphiphilic block copolymer is biodegradable polyester-block-methoxy polyethylene glycol (MePEG).

In one or more embodiments of the preparation method, the amphiphilic block copolymer is a triblock copolymer.

In one or more embodiments of the preparation method, the amphiphilic block copolymer is biodegradable polyester-block-polyethylene glycol (PEG)-block-polyester.

In one or more embodiments of the preparation method, the polyester block is selected from poly(D,L-lactide), poly(L-lactide), poly(D-lactide), polyglycolide, poly(ε-caprolactone), and copolymers and mixtures thereof.

In one or more embodiments of the preparation method, the polyester block is poly(D,L-lactide) (PDLLA).

In one or more embodiments of the preparation method, the one or more pharmaceutically acceptable excipients, carriers or diluents are selected from the group consisting of buffering agents, osmolality agents, cryopreservation protectants, lyophilization protectants, bulking agents, surfactants, and solvents.

In one or more embodiments of the preparation method, the molecular weight of PDLLA is within the range from about 750 to about 50,000.

In one or more embodiments of the preparation method, the molecular weight of MePEG is within the range from about 750 to about 20,000.

In one or more embodiments of the preparation method, the molecular weight of PEG is within the range from about 200 to about 20,000.

In one or more embodiments of the preparation method, the preparation method further comprises storing the frozen polymeric micellar cinacalcet at about −20° C. for long term storage.

In one or more embodiments of the preparation method, the preparation method further comprises storing the lyophilized polymeric micellar cinacalcet at about 2° C. to about 8° C. for long term storage.

In one or more embodiments of the preparation method, the preparation method further comprises storing the cinacalcet polymer ethanol solution, cinacalcet/polymer matrix, or lyophilized polymeric micellar cinacalcet at about 2° C. to about 8° C. for long term storage

In one or more embodiments of the preparation method, the preparation method further comprises reconstituting the lyophilized polymeric micellar cinacalcet prior to administration

In one or more embodiments of the preparation method, the preparation method further comprises reconstituting the cinacalcet polymer ethanol solution, cinacalcet/polymer matrix, or lyophilized polymeric micellar cinacalcet prior to administration

One exemplary method of making polymeric micellar cinacalcet compositions is to dissolve cinacalcet HCl and the diblock copolymer in a biocompatible solvent, such as ethanol. Diluting in aqueous solution resulting in cinacalcet micellar formulation. The solvent can also be removed leaving cinacalcet/polymer matrix for increased stability. In the case of solvent removal, various solvents, such as acetone, acetonitrile, DCM can be selected, as they are eventually removed alleviating safety concerns. The cinacalcet/polymer matrix can be dissolved in aqueous solution followed by lyophilization for faster reconstitution. This method is particularly suitable for water-soluble diblock copolymers.

Another exemplary method of making polymeric micellar cinacalcet compositions is to dissolve cinacalcet and copolymer in an organic solvent, such as acetone, and then mix the resulting solution with an aqueous buffer solution. The solvent is removed by evaporation. Micellar and even nanoparticle formulations can be obtained after solvent evaporation. A cryo- and/or lyophilization protectant is added to prevent aggregation and phase separation of the micelle formulations upon freezing and lyophilization. A surfactant can be added to further reduce chances of aggregation. The micelle formulations are lyophilized for long-term storage stability. The lyophilized formulations form micellar cinacalcet upon reconstitution with an aqueous solution or water.

In yet another aspect, the present application generally relates to use of the pharmaceutical composition disclosed herein for controlling PTH levels.

In yet another aspect, the present application generally relates to use of the pharmaceutical composition disclosed herein for regulating calcium and phosphorus in blood.

In yet another aspect, the present application generally relates to use of the pharmaceutical composition disclosed herein for treating primary hyperparathyroidism.

In yet another aspect, the present application generally relates to use of the pharmaceutical composition disclosed herein for treating secondary hyperparathyroidism (e.g., in patients with chronic kidney disease on dialysis).

In yet another aspect, the present application generally relates to use of the pharmaceutical composition disclosed herein for treating hypercalcemia (e.g., in patients with parathyroid carcinoma).

In yet another aspect, the present application generally relates to the pharmaceutical composition disclosed herein for use in controlling parathyroid hormone (PTH) levels.

In yet another aspect, the present application generally relates to the pharmaceutical composition disclosed herein for use in regulating calcium and phosphorus in blood.

In yet another aspect, the present application generally relates to the pharmaceutical composition disclosed herein for use in treating primary hyperparathyroidism.

In yet another aspect, the present application generally relates to the pharmaceutical composition disclosed herein for use in treating secondary hyperparathyroidism.

In yet another aspect, the present application generally relates to the pharmaceutical composition disclosed herein for use in treating parathyroid carcinoma. In yet another aspect, the present application generally relates to use of cinacalcet, or a pharmaceutically acceptable salt thereof, and at least one amphiphilic block copolymer in the manufacture of a medicament.

In one or more embodiments, the medicament is used for controlling parathyroid hormone (PTH) levels.

In one or more embodiments, the medicament is used for regulating calcium and phosphorus in blood.

In one or more embodiments, the medicament is used for treating primary hyperparathyroidism.

In one or more embodiments, the medicament is used for treating secondary hyperparathyroidism.

In one or more embodiments, the medicament is used for treating parathyroid carcinoma.

Those skilled in the art will recognize if a stereo-center exists in a compound described herein, stereoisomers and all optical isomers of the compound (e.g., R and S enantiomers), as well as racemic, diastereomeric and other mixtures of such isomers are within the scope of the present application. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are contemplated by the present invention. Those of ordinary skill in the art will readily appreciate that analogous ratios are contemplated for more complex isomer mixtures.

Isotopically-labeled compounds are also within the scope of the present disclosure. As used herein, an “isotopically-labeled compound” refers to a presently disclosed compound including pharmaceutical salts and prodrugs thereof, each as described herein, in which one or more atoms are replaced by an atom having an atomic mass or mass number different from the atomic mass or mass number usually found in nature. Examples of isotopes that can be incorporated into compounds presently disclosed include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

By isotopically-labeling the presently disclosed compounds, the compounds may be useful in drug and/or substrate tissue distribution assays. Tritiated (³H) and carbon-14 (¹⁴C) labeled compounds are particularly preferred for their ease of preparation and detectability. Further, substitution with heavier isotopes, such as deuterium (²H) can afford certain therapeutic advantages resulting from greater metabolic stability, for example increased in vivo half-life or reduced dosage requirements and, hence, may be preferred in some circumstances. Isotopically labeled compounds presently disclosed, including pharmaceutical salts, esters, and prodrugs thereof, can be prepared by any means known in the art.

Further, substitution of normally abundant hydrogen (H) with heavier isotopes, such as deuterium can afford certain therapeutic advantages, e.g., resulting from improved absorption, distribution, metabolism and/or excretion (ADME) properties, creating drugs with improved efficacy, safety, and/or tolerability. Benefits may also be obtained from replacement of normally abundant ¹²C with ¹³C. (See, WO 2007/005643, WO 2007/005644, WO 2007/016361, and WO 2007/016431.)

Compounds of the present invention are, subsequent to their preparation, preferably isolated and purified to obtain a composition containing an amount by weight equal to or greater than 95% (“substantially pure”), which is then used or formulated as described herein. In one or more embodiments, the compounds of the present invention are not less than 98% pure.

Solvates and polymorphs of the compounds of the present application are also contemplated herein. Solvates of the compounds of the present invention include, for example, hydrates.

The compositions of the present application may also contain adjuvants, such as preservatives. Prevention of the action of microorganisms may be ensured by the inclusion of various antibacterial and antifungal agents, for example, paragen, chlorobutanol, phenol sorbic acid, and the like. It may also be desirable to include isotonic agents, such as sugars, sodium chloride, and the like.

Administering a compound, or a pharmaceutically acceptable salt thereof, or a pharmaceutical composition described herein to a mammal comprises any suitable delivery method. Most suitable means of administration for a particular patient will depend on the nature and severity of the disease or condition being treated or the nature of the therapy being used and on the nature of the active compound.

Useful dosages of a compound described herein can be determined by comparing their in vitro activity and in vivo activity in animal models. Methods for the extrapolation of effective dosages in mice, and other animals, to humans are known to the art; for example, see U.S. Pat. No. 4,938,949, which is incorporated by reference in its entirety.

The amount of a compound described herein, required for use in treatment can vary not only with the particular salt selected but also with the route of administration, the nature of the condition being treated and the age and condition of the patient and can be ultimately at the discretion of the attendant physician or clinician. In general, total daily dose of the compositions of the present application to be administered to a human or other mammal host in single or divided doses may be in amounts, for example, from about 0.05 mg/kg to about 8 mg/kg body weight (e.g., from about 0.1 mg/kg to about 8 mg/kg, from about 0.1 mg/kg to about 5 mg/kg, from about 0.1 mg/kg to about 2 mg/kg).

Exemplary pharmaceutical dosage forms for injection or infusion can include sterile aqueous solutions or dispersions or sterile powders comprising the active ingredient which are adapted for the extemporaneous preparation of sterile injectable or infusible solutions or dispersions. In all cases, the ultimate dosage form should be sterile, fluid and stable under the conditions of manufacture and storage.

The disclosed method can include a kit comprising a compound, or a pharmaceutically acceptable salt thereof, or a pharmaceutical composition described herein and instructional material which can describe administering a compound, or a pharmaceutically acceptable salt thereof, or a composition described herein to a subject. This should be construed to include other embodiments of kits that are known to those skilled in the art, such as a kit comprising a (such as sterile) solvent for dissolving or suspending a compound, or a pharmaceutically acceptable salt thereof, or a composition described herein prior to administering a compound, or a pharmaceutically acceptable salt thereof, or a composition described herein to a cell or a subject. In some embodiments, the subject can be a human.

Materials, compositions, and components disclosed herein can be used for, can be used in conjunction with, can be used in preparation for, or are products of the disclosed methods and compositions. It is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

### EXAMPLES

The following examples are given for the purpose of illustrating the present application, but not for limiting the scope or spirit of the present application.

Synthesis of Biodegradable Polyester-block-MePEG Diblock Copolymers (e.g., PDLLA-MePEG) and Polyester-block-PEG-block-polyester Triblock Copolymers (e.g., PDLLA-PEG-PDLLA) of Various Compositions

Biodegradable polyester-block-methoxy polyethylene glycol (MePEG) diblock copolymers or polyester-block-PEG-block-polyester triblock copolymers are synthesized via ring-opening polymerization of monomers, such as D,L-lactide, D-lactide, L-lactide, glycolide, and ε-caprolactone, in presence of MePEG or PEG and stannous octoate. The hydroxy group of MePEG or PEG acts as an initiator for the ring-opening polymerization. Hence, it is attached to the growing polyester chain, and forms polyester-MePEG diblock or polyester-PEG-polyester triblock polymers. Stannous octoate acts as catalyst. The monomers or their mixtures, MePEG or PEG of various molecular weight, and stannous octoate are added to a clean flask with stir. The flask is sealed and heated to 120° C. to 160° C. for bulk melt polymerization with stirring. The polymerization typically completes within 72 hours. Before polymerization, impurities in monomers can be removed via recrystallization. Moistures in MePEG or PEG can be removed by vacuuming.

The type of monomers, the molecular weight of MePEG or PEG, and the ratio of monomer to MePEG or PEG can be adjusted for desired polymer composition and properties. The expected molecular weight of the polyester block is calculated as: weight ratio of monomer to MePEG x molecular weight of MePEG, or ½ weight ratio of monomer to PEG x molecular weight of PEG.

### Example 1a

To a 250 mL clean flask containing a magnetic stir, 75 g of D,L-lactide (DLLA), 25 g of MePEG with molecular weight of 5000, and 0.2 g stannous octoate were added. The flask was sealed and immersed in an oil bath heated to 135° C. The content was stirred with temperature maintained at 125-150° C. for about 8 hours. The polymerization was quenched by cooling to room temperature and the diblock copolymer was obtained.

### Example 1b

To a 10 mL clean flask containing a magnetic stir, 6 g of PEG with molecular weight of 3350 was added. The flask was immersed in a metal sand bath heated to about 140° C. to melt the PEG. With stirring, vacuum was applied for 2 hours to remove residual moisture in PEG. 4 g of D,L-lactide was added. After molten, 0.03 g stannous octoate was added. Brief vacuuming was applied after each opening of the flask to remove moisture, followed by sealing of the flask. The content was stirred with temperature maintained at about 140° C. for about 17 hours. The polymerization was quenched by cooling to room temperature and the triblock copolymer was obtained.

The polymer in Example 1a is named as PDLLA-MePEG 15-5, representing poly(D,L-lactide) (PDLLA) and MePEG diblocks and their expected molecular weights of 15 k for PDLLA block and 5 k for MePEG block. The polymer in Example 1b is named as PDLLA-PEG-PDLLA 1.1-3.35-1.1, representing poly(D,L-lactide) (PDLLA) and PEG triblocks, with their expected molecular weights of 1.1 k for PDLLA block and 3.35 k for PEG block. Table 1 provides examples of various block polymers synthesized via this approach. Table 2 provides additional examples of various block polymers synthesized with molecular weights, molecular weight distributions, and oligomer contents determined by ¹H-NMR, GPC, and RP-HPLC, respectively. An example GPC chromatogram is shown in FIG. 2.

TABLE 1
Diblock and triblock copolymers and their properties

| Di-block copolymers | Soluble or insoluble in water |
|---|---|
| PDLLA-MePEG 1.3-1.9 | Soluble |
| PDLLA-MePEG 2.1-5 | Soluble |
| PDLLA-MePEG 5-5 | Borderline soluble |
| PDLLA-MePEG 11.7-5 | Insoluble |
| PDLLA-MePEG 15-5 | Insoluble |
| PDLLA-MePEG 20-5 | Insoluble |
| PDLLA-MePEG 30-5 | Insoluble |
| PCL-MePEG 5-5 | Borderline soluble |
| PCL-MePEG 11.7-5 | Insoluble |
| PDLLA-PEG-PDLLA 1.1-3.35-1.1 | soluble |
| PDLLA-PEG-PDLLA 1.3-6-1.3 | soluble |

TABLE 2
Molecular weight and polydispersity of the synthesized PLA-
MePEG diblock copolymers and PLA-PEG-PLA triblock copolymers

| | | | MW of | MW of | | |
|---|---|---|---|---|---|---|
| | | Polymer | PLA | MePEG | Polydis- | % of |
| Block copolymer | Lot number | MW^{a} | block^{a} | block^{a} | persity^{b} | Oligomers^{c} |
| PDLLA-MePEG | NB007-031 | 3572 | 1332 | 2240 | 1.13 | 3.36 |
| 2-1.3 | | | | | | |
| PDLLA-MePEG | NB009-001 | 3620 | 1519 | 2101 | 1.11 | 2.33 |
| 2-1.6 | | | | | | |
| PDLLA-MePEG 2-2 | NB007-028 | 4161 | 2001 | 2160 | 1.17 | 2.30 |
| PDLLA-MePEG 2-2 | NB009-029 | 4199 | 2015 | 2184 | 1.06 | 3.85 |
| PDLLA-PEG-PDLLA | NB013-086-2 | 4452^{d} | NT | NT | 1.09 | 2.72 |
| 1.1-3.35-1.1 | | | | | | |
| PDLLA-PEG-PDLLA | NB013-086-3 | 6793^{d} | NT | NT | 1.08 | 0.77 |
| 1.3-6-1.3 | | | | | | |

^{a}determined by H-NMR except triblock copolymers
^{b}polydispersity is ratio of weight average molecular weight over number average molecular weight as determined by GPC using THF as mobile phase and PEGs as standards
^{c}determined by RP-HPLC
^{d}number average molecular weight determined by GPC using THF as mobile phase and PEGs as standards

### Preparation of Block Copolymer Cinacalcet Solution (Method A) or Matrix (Method C) for Micelle Formation and Lyophilization

The polymeric micellar cinacalcet formulations can be prepared by dissolving cinacalcet HCl and block copolymers in biocompatible solvents, followed by diluting in an aqueous solution, such as saline or water.

### Example 2 (Table 3, Formulation L)

2.4 g PDLLA-MePEG 5-5 was added to 5.6 mL ethanol and warmed to 40-50° C., resulting in a polymer solution. 0.27 g cinacalcet HCl was then added to the polymer solution, giving a cinacalcet/polymer ethanol solution. 1 to 10 dilution of the polymer solution in saline resulted in a colloid solution, with 15× diluted turbidity of 42.5 NTU.

### Example 3 (Table 3, Formulation I)

3.4 g PDLLA-MePEG 1.3-1.9 was added to 7.9 mL ethanol and warmed to about 50° C., resulting in a polymer solution. 0.42 g cinacalcet HCl was then added to the polymer solution, giving a cinacalcet/polymer ethanol solution. 1 to 10 dilution of the polymer solution in saline resulted in a clear solution.

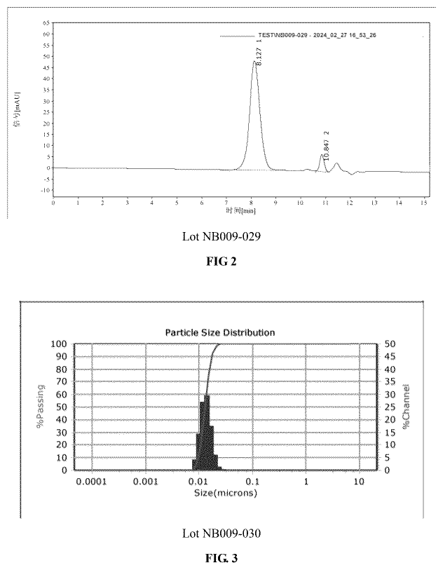

Table 3, Table 4a and Table 4b provide compositions evaluated using the above approach. Note that the organic solvent ethanol in the formulations can be removed to obtain cinacalcet/copolymer matrix, for higher stability for long-term storage. The cinacalcet/polymer matrix can be dissolved in aqueous solution and lyophilized for rapid reconstitution. Table 5 provides exemplary formulations prepared by lyophilization. The described method is particularly suitable for water-soluble diblock copolymers. FIG. 3 provides a typical example of particle size distributions of cinacalcet polymer micelles, as measured by DLS.

TABLE 3
Evaluating preparation methods of polymeric micellar cinacalcet formulations-dissolving cinacalcet
HCl and block copolymers in biocompatible solvents followed by diluting with water or saline

| | | | | Drug | | Appearance when |
|---|---|---|---|---|---|---|
| | | | Drug | conc, | | diluted in aqueous |
| Formulation | Polymer | Solvent | loading | mg/ml | Preparation | solution |
| A | PDLLA-MePEG | ethanol | 6.3% | 6.7 | Cinacalcet HCl | Clear to colloidal |
| | 1.3-1.9 | | | | and polymer in | solutions were |
| C | PDLLA-MePEG | ethanol | 4.4% | 6.7 | solvent(s) were | obtained upon |
| | 1.3-1.9 | | | | dissolved, with | dilution in water |
| D | PDLLA-MePEG | ethanol | 3.3% | 6.7 | or without | or saline. |
| | 1.3-1.9 | | | | gentle heat. | |
| E | PDLLA-MePEG | ethanol | 3.3% | 6.7 | | |
| | 1.3-1.9 | | | | | |
| F | PDLLA-MePEG | ethanol | 3.3% | 6.7 | | |
| | 1.3-1.9 | | | | | |
| G | PDLLA-MePEG | ethanol | 3.3% | 6.7 | | |
| | 1.3-1.9 | | | | | |
| H | PDLLA-MePEG | propylene | 11.1% | 35.9 | | |
| | 1.3-1.9 | glycol | | | | |
| I | PDLLA-MePEG | ethanol | 11.1% | 35.9 | | |
| | 1.3-1.9 | | | | | |
| K | PDLLA-MePEG | ethanol | 5.3% | 16.7 | | |
| | 2.1-5 | | | | | |
| L | PDLLA-MePEG 5-5 | ethanol | 10.1% | 32.6 | | |
| M | PDLLA-MePEG 5-5 | ethanol | 4.8% | 14.9 | | |
| N | PDLLA-MePEG | ethanol | 4.8% | 14.8 | | |
| | 1.3-1.9 | | | | | |
| W | PDLLA-MePEG | 1:2 | 10.2% | 10.7 | | |
| | 20-5 | NMP:ethanol | | | | |

TABLE 4a
Cincacalcet polymeric micelle-ethanol solution formulations (dissolving cinacalcet
HCl and block copolymers in ethanol followed by diluting with water)

| | Polymer type | | Cinacalcet | 10x dilution in water | |
|---|---|---|---|---|---|
| | PDLLA-MePEG or | Cinacalcet | concentration^{a}, | Appearance - | |
| Formulation ID | PDLLA-PEG-PDLLA | loading, % | mg/mL | turbidity (NTU) | pH |
| NB009-004 | 2-2 | 10% | 20 | Clear - 1.2 | 4.14 |
| NB009-004 | 2-1.6 | 10% | 20 | Clear - 0.7 | 3.88 |
| NB009-004 | 2-1.3 | 10% | 20 | Clear - 0.6 | 3.95 |
| NB009-004 | 2-2 | 15% | 30 | Clear - 0.6 | 4.07 |
| NB009-004 | 2-1.6 | 15% | 30 | Clear - 1.2 | 3.80 |
| SP-240708^{b} | 1.1-3.35-1.1 | 10% | 30 | Colloidal | 5.07 |
| SP-240708^{b} | 1.3-6-1.3 | 10% | 30 | Colloidal | 5.00 |
| SP-240708^{b} | 1.1-3.35-1.1 | 15% | 45 | Colloidal | 5.11 |
| SP-240708^{b} | 1.3-6-1.3 | 15% | 45 | Colloidal | 5.00 |

^{a}cinacalcet concentration in ethanol
^{b}containing NaOAc about 20 mg/mL

TABLE 4b
Cincacalcet polymeric micelle-ethanol solution formulations (dissolving cinacalcet
HCl and block copolymers in ethanol followed by diluting with D5W)

| | Polymer type | | Cinacalcet | 10x dilution in D5W | |
|---|---|---|---|---|---|
| | PDLLA-MePEG or | Cinacalcet | concentration^{a}, | Appearance - | |
| Formulation ID | PDLLA-PEG-PDLLA | loading, % | mg/mL | turbidity (NTU) | pH |
| NB010-004 | 2-2 | 10% | 20 | Clear - 0.8 | 4.17 |
| NB010-004 | 2-1.6 | 10% | 20 | Clear - 1.0 | 4.04 |
| NB010-004 | 2-1.3 | 10% | 20 | Clear - 0.9 | 4.06 |
| NB010-004 | 2-2 | 15% | 30 | Clear - 1.5 | 4.11 |
| NB010-004 | 2-2 | 15% | 30 | Clear - 0.5 | 3.92 |
| SP-240708^{b} | 1.1-3.35-1.1 | 10% | 30 | Colloidal | 5.06 |
| SP-240708^{b} | 1.3-6-1.3 | 10% | 30 | Colloidal | 4.98 |
| SP-240708^{b} | 1.1-3.35-1.1 | 15% | 45 | Colloidal | 5.08 |
| SP-240708^{b} | 1.3-6-1.3 | 15% | 45 | Colloidal | 4.96 |

^{a}cinacalcet concentration in ethanol
^{b}containing NaOAc about 20 mg/mL

TABLE 5
Cinacalcet polymeric micelle-lyophilized^{b} formulations (dissolving cinacalcet HCl and block copolymers in tert-butanol
or ethanol, removing ethanol, diluting in an aqueous solution, and lyophilizing to remove water or alcohol)

| | PDLLA- | | | | | | Recon^{d} | |
|---|---|---|---|---|---|---|---|---|
| | MePEG or | | | | Cinacalcet | | turbidity, | Recon^{d} |
| | PDLLA- | Cinacalcet | | | conc., | Recon^{c} | NTU | pH |
| Formulation ID | PEG-PDLLA | loading, % | Solvent^{a} | Diluent | mg/mL | appearance | (t = 0) | (t = 0) |
| NB010-006#16 | 2-2 | 15% | tert-butanol | 100 mM NaOAc | 10.0 | clear solution | 10.1 | 5.79 |
| NB010-006#17 | 2-1.7 | 15% | tert-butanol | 100 mM NaOAc | 10.0 | clear solution | 13.8 | 5.76 |
| NB010-006#20 | 2-1.7 | 15% | tert-butanol | 100 mM NaOAc + | 10.0 | clear solution | 10.7 | 5.81 |
| | | | | 3% w/v mannitol | | | | |
| NB010-006#22 | 2-2 | 15% | ethanol | 100 mM NaOAc + | 10.0 | clear solution | 10.1 | 5.55 |
| | | | | 3% w/v mannitol | | | | |
| NB010-006#23 | 2-1.7 | 15% | ethanol | 100 mM NaOAc + | 10.0 | clear solution | 6.6 | 5.48 |
| | | | | 3% w/v mannitol | | | | |
| NB009-010#25 | 2-2 | 10% | ethanol | 100 mM NaOAc | 10.0 | clear solution | 13.0 | 5.84 |
| NB009-010#26 | 2-1.7 | 10% | ethanol | 100 mM NaOAc | 10.0 | clear solution | 7.2 | 5.63 |
| NB009-010#27 | 2-1.3 | 10% | ethanol | 100 mM NaOAc | 10.0 | clear solution | 4.9 | 5.87 |
| NB010-010#30 | 2-1.3 | 10% | ethanol | titrate using 1M | 10.0 | clear solution | 3.7 | 5.64 |
| | | | | NaOAc to pH 5.0 | | | | |
| NB010-010#31 | 2-1.3 | 10% | ethanol | to 100 mM NaOAc | 10.0 | clear solution | 4.6 | 5.75 |
| NB010-010#34 | 2-1.3 | 10% | ethanol | titrate using 1M | 10.0 | clear solution | 2.5 | 5.58 |
| | | | | NaOAc to pH 5.0 | | | | |
| NB010-010#35 | 2-1.3 | 10% | ethanol | 100 mM NaOAc | 10.0 | clear solution | 2.7 | 5.74 |
| NB010-020#36 | 2-1.3 | 10% | ethanol | 100 mM NaOAc + | 10.0 | clear solution | 8.3 | 5.95 |
| | | | | 0.1% PS80 | | | | |
| NB010-020#38 | 2-1.3 | 10% | ethanol | 50 mM Na citrate | 10.0 | clear solution | 7.3 | 6.11 |
| NB009-160-SC | 2-2 | 10% | ethanol | 50 mM Na citrate | 10.0 | clear solution | 2.9 | 5.58 |
| NB009-160-MT | 2-2 | 10% | ethanol | 100 mM NaOAc + | 5.0 | clear solution | 4.2 | 5.9 |
| | | | | 5% w/v Mannitol | | | | |
| NB009-164-3 | 2-2 | 10% | ethanol | 100 mM NaOAc + | 10.0 | clear solution | 3.2 | 5.48 |
| | | | | 5% w/v Mannitol | | | | |
| NB009-164-4 | 2-2 | 10% | ethanol | 100 mM NaOAc + | 10.0 | clear solution | 4.8 | 5.78 |
| | | | | 10% w/v Mannitol | | | | |
| NB009-030 | 2-2 | 10% | ethanol | 100 mM NaOAc | 10.0 | clear solution | 3.9 | 5.31 |
| NB009-099 | 2-2 | 10% | ethanol | 100 mM NaOAc | 10.0 | clear solution | 4.2 | 5.05 |
| NB009-176 | 2-2 | 10% | ethanol | 100 mM NaOAc + | 10.0 | clear solution | 3.8 | 5.34 |
| | | | | 10% w/v Mannitol | | | | |

^{a}A cinacalcet/polymer/ethanol solution ((cinacalcet + polymer):ethanol = 1:2 w/v) was prepared, followed by removal of ethanol using vacuum and modest heat (about 45° C.), and then reconstitution of the cinacalcet/polymer matrix with diluent to cinacalcet concentration of 10 mg/mL, or 5 mg/mL in the case of NB009-160-MT. In the case of cinacalcet/polymer/tert-butanol solution ((cinacalcet + polymer):tert-butanol = 1:4 w/v), the diluent was added directly to prepare a 10 mg/mL cinacalcet solution.
^{b}A 2 mL of the reconstituted cinacalcet solution from above was added to a glass vial and lyophilized to remove water.
^{c}The lyophilized formulation was added with 1.85 mL water, to obtain reconstituted cinacalcet solution at 5 or 10 mg/mL concentration.
^{d}The above reconstituted 10 mg/mL cinacalcet solution was diluted 5x with NS or D5W for turbidity and pH measurements.

### Preparation of Cinacalcet Micelles (Method B) and Lyophilization

Cinacalcet polymeric micelles can also be prepared by dissolving both cinacalcet HCl and copolymer in an organic solvent (such as acetone, acetonitrile, and DCM), mixing with an aqueous phase, and then removing the organic solvent. This method is particularly suitable for water-insoluble copolymers as well. The preferred solvent is acetone due to its low toxicity, high solubility for both cinacalcet HCl and copolymers, and high volatility. However, during solvent evaporation, cinacalcet HCl tends to form crystals. Hence, the optimal formulation and process conditions need to be developed for easy manufacturing.

Table 6 provides compositions and methods evaluated during screening. Note that a formulation with a higher drug loading and drug concentration (e.g., at or above drug loading of 10% and cinacalcet concentration of 5 mg/mL) is preferred. PLA and PLGA are preferred over PCL due to their proven biocompatibility. None of the formulations studied in Table 3 achieved drug loading of 10% and concentration of 5 mg/mL, except using PCL as a hydrophobic block. It is speculated that that PCL is more hydrophobic, which can contribute to a higher drug payload and concentration.

TABLE 6
Evaluating preparation methods of polymeric micellar cinacalcet formulations-
dissolving cinacalcet HCl and diblock copolymers in solvents, mixing with
an aqueous solution, and then removing solvent by evaporation

| | | | | | Drug | |
|---|---|---|---|---|---|---|
| Formu- | | Organic | Aqueous | Drug | conc., | |
| lation | Polymer | solvents | phase | loading | mg/ml | Observations |
| AB | PCL-MEPEG | acetone | water | 5% | 1.5 | clear colloid |
| | 11.7-5 | | | | | |
| AC | PCL-MEPEG | acetone | water | 10% | 1.1 | clear colloid |
| | 11.7-5 | | | | | |
| AE | PCL-MEPEG 5-5 | acetone | water | 10% | 3.3 | clear colloid |
| AF | PCL-MEPEG 5-5 | acetone | 20 mM PBS | 10% | 5.9 | hazy colloid |
| | | | pH 6.8 | | | |
| AG | PCL-MEPEG 5-5 | acetone | 80 mM PBS | 10% | 5.7 | colloid |
| | | | pH 6.7 | | | |
| AI | PDLLA-MePEG | acetone | water | 5% | 2.5 | clear colloid |
| | 11.7-5 | | | | | |
| AJ | PDLLA-MePEG | Acetone: and | water | 5% | 6.0 | clear colloid |
| | 11.7-5 | ethanol (1:1) | | | | |
| AL | PDLLA-MePEG | acetone | 100 mM NaOAc | 30% | 1.0 | clear colloid |
| | 20-5 | | pH 6.2 | | | |
| AN | PDLLA-MePEG | acetone | water | 30% | 1.0 | clear colloid |
| | 20-5 | | | | | |
| AO | PDLLA-MePEG | acetone | water | 50% | 1.0 | clear colloid |
| | 20-5 | | | | | |
| AQ | PDLLA-MePEG | acetone | water | 70% | 1.0 | clear colloid |
| | 20-5 | | | | | |
| BH | none | acetone | 20 mM HOAc, | 8.3% | 5.0 | clear solution |
| | | | 6% HPβCD | | | |

To achieve the desired drug loading and concentration, it has been discovered that acetate buffer assists in preventing crystallization of cinacalcet HCl during solvent evaporation. Presence of cyclodextrins, such as hydroxypropyl beta cyclodextrin (HPBCD) further increases drug loading.

Table 7 provides further studied formulations that achieved the drug loading of 10% and 5 mg/mL or above. The studied compositions and preparation methods also demonstrate robustness.

Based on the results in Table 7, the following observations can be made. The sodium acetate (NaOAc) buffer with a concentration at 80-100 mM has a better capacity in preventing drug crystallization during evaporation of acetone. PDLLA-MePEG has a higher drug-carrying capacity than PLGA-MePEG, probably due to its higher hydrophobicity. Addition of HPBCD can further increase drug loading capacity. Addition of a minor amount of surfactant, such as polysorbate 80 (PS 80) and Pluronic F68 can reduce potential aggregation.

### Example 4 (Table 7 Formulation BM)

2 mL of 100 mM sodium acetate, 2 mL of 300 mg/mL HPBCD, and 6 mL water were mixed to form an aqueous solution. 3.3 mL of 30 mg/mL cinacalcet HCl acetone and 2 mL of 200 mg/mL PDLLA-MePEG 20-5 acetone were mixed to form an organic solution. The aqueous and organic solutions were combined to form a clear solution, stirred for 20 hours to allow evaporation of acetone. The solution was QS to 10 mL with water. A colloidal solution was obtained with drug loading at 20%, drug concentration at 10 mg/mL, and pH at 4.5.

### Example 5 (Table 7 Formulation DO)

0.56 g cinacalcet HCl, 5.01 g PDLLA-MePEG 15-5, and 70 mL acetone were combined to make an organic solution. The above solution was added to 100 mL of 0.1M NaOAc in 0.01% PS80 aqueous solution with pH 7.9 with magnetically stirring (about 400-500 rpm). Acetone was evaporated in about 20 hours, resulting in colloidal formulation of total weight of 85.5 g (evaporation of smaller amount of water also occurred). The colloidal formulation was then added with 6 g HPBCD and QS to 100 mL with water.

### Example 6 (Table 7 Formulation DW)

1.17 g cinacalcet HCl, 10 g PDLLA-MePEG 15-5, and 120 mL acetone were combined to make an organic solution. The above solution was added to 200 mL of 0.1M NaOAc aqueous solution with pH 7.8 with magnetic stirring. Acetone was evaporated in about 36 hours, resulting in colloidal formulation of 201 mL, with pH 5.1 and 1:15 dilution turbidity at 12.3 NTU.

TABLE 7
Evaluating preparation methods of polymeric micellar cinacalcet formulations by
solvent evaporation-effects of buffer, cryo- and lyophilization protectants.

| | | | Cinacalcet | Cinacalcet | |
|---|---|---|---|---|---|
| | | | HCl | base | |
| Formulation | | | loading, | conc, | |
| code | Polymer | Aqueous phase | % | mg/ml | Appearance |
| BC | PDLLA-MePEG | 100 mM NaOAc pH 6.0 | 10.2% | 4.6 | clear colloid |
| | 11.7-5 | | | | |
| BD | PDLLA-MePEG | 20 mM NaOAc pH 6.0 | 10.2% | 4.6 | colloid |
| | 11.7-5 | | | | |
| BE | PDLLA-MePEG | 20 mM HOAc | 10.2% | 4.4 | colloid |
| | 11.7-5 | | | | |
| BF | PDLLA-MePEG | 6% HPβCD 20 mM NaOAc pH | 19.8% | 8.9 | clear colloid |
| | 11.7-5 | 6.0 | | | |
| BJ | PDLLA-MePEG | 6% HPβCD 20 mM NaOAc pH | 19.8% | 8.9 | colloid |
| | 11.7-5 | 4.4 | | | |
| BL | PDLLA-MePEG | 6% sucrose 20 mM NaOAc pH | 11.2% | 5.1 | colloid |
| | 11.7-5 | 6.0 | | | |
| BM | PDLLA-MePEG 20-5 | 6% HPβCD 20 mM NaOAc pH | 20.0% | 8.9 | colloid |
| | | 6.0 | | | |
| BR | PDLLA-MePEG 20-5 | 100 mM NaOAc pH 6.0 | 11.2% | 5.1 | clear colloid |
| BS | PDLLA-MePEG 20-5 | 6% sucrose 80 mM NaOAc pH | 11.2% | 5.1 | colloid |
| | | 6.0 | | | |
| BU | PDLLA-MePEG | 3% HPβCD 20 mM NaOAc pH | 11.2% | 5.1 | colloid |
| | 11.7-5 | 4.4 | | | |
| BW | PDLLA-MePEG 20-5 | 6% HPβCD 80 mM NaOAc pH | 11.2% | 5.1 | colloid |
| | | 6.0 | | | |
| BY | PDLLA-MePEG | 6% HPβCD 100 mM NaOAc | 20.2% | 10.3 | colloid |
| | 11.7-5 | pH 6.0 | | | |
| BZ | PDLLA-MePEG 20-5 | 6% HPβCD 100 mM NaOAc | 20.2% | 10.3 | colloid |
| | | pH 6.0 | | | |
| CA | PDLLA-MePEG | 6% HPβCD 100 mM NaOAc | 20.2% | 10.3 | colloid |
| | 11.7-5 | pH 4.4 | | | |
| CB | PDLLA-MePEG 20-5 | 6% HPβCD 100 mM NaOAc | 20.2% | 10.3 | colloid |
| | | pH 4.4 | | | |
| CC | PDLLA-MePEG | 6% HPβCD 100 mM NaOAc | 11.2% | 5.1 | colloid |
| | 11.7-5 | pH 6.0 | | | |
| CD | PDLLA-MePEG 20-5 | 6% HPβCD 100 mM NaOAc | 11.2% | 5.1 | colloid |
| | | pH 6.0 | | | |
| CE | PDLLA-MePEG | 6% HPβCD 100 mM NaOAc | 11.2% | 5.1 | colloid |
| | 11.7-5 | pH 4.4 | | | |
| CF | PDLLA-MePEG 20-5 | 6% HPβCD 100 mM NaOAc | 11.2% | 5.1 | colloid |
| | | pH 4.4 | | | |
| CG | PDLLA-MePEG | 6% HPβCD 100 mM NaOAc | 20.0% | 10.1 | colloid |
| | 11.7-5 | pH 4-6 | | | |
| CH | PDLLA-MePEG 15-5 | 6% HPβCD 100 mM NaOAc | 20.0% | 9.9 | clear colloid |
| | | pH 4.4 | | | |
| CI | PDLLA-MePEG | 6% HPβCD 100 mM NaOAc | 20.0% | 9.9 | clear colloid |
| | 11.7-5 | pH 4.4 | | | |
| CJ | PDLLA-MePEG 15-5 | 6% HPβCD 100 mM NaOAc | 11.1% | 5.0 | colloid |
| | | pH 4.4 | | | |
| CK | PDLLA-MePEG | 6% HPβCD 100 mM NaOAc | 11.1% | 5.0 | colloid |
| | 11.7-5 | pH 4.4 | | | |
| CL | PDLLA-MePEG | 100 mM NaOAc pH 4.4 | 11.2% | 5.1 | colloid |
| | 11.7-5 | | | | |
| CM | PDLLA-MePEG 15-5 | 100 mM NaOAc pH 4.4 | 11.1% | 5.0 | clear colloid |
| CO | PDLLA-MePEG 15-5 | 100 mM NaOAc pH 4.4 | 11.3% | 5.0 | clear colloid |
| CP | PDLLA-MePEG | 100 mM NaOAc pH 4.4 | 12.0% | 5.4 | clear colloid |
| | 11.7-5 | | | | |
| CR | PDLLA-MePEG 15-5 | 100 mM NaOAc pH 4.4 | 13.0% | 5.9 | hazy colloid |
| CS | PDLLA-MePEG 15-5 | 100 mM NaOAc pH 4.4 | 11.6% | 5.2 | clear colloid |
| CT | PDLLA-MePEG 15-5 | 100 mM NaOAc pH 4.4 | 11.6% | 5.2 | clear colloid |
| CV | PDLLA-MePEG 30-5 | 6% HPβCD 100 mM NaOAc | 11.6% | 5.2 | translucent |
| | | pH 4.4 | | | colloid |
| CY | PDLLA-MePEG 15-5 | 6% sucrose 100 mM NaOAc | 11.6% | 5.2 | clear colloid |
| | | pH 4.4 | | | |
| DB | PDLLA-MePEG 15-5 | 6% sucrose 100 mM NaOAc | 11.6% | 5.2 | clear colloid |
| | | pH 4.4 | | | |
| DC | PDLLA-MePEG 15-5 | 6% sucrose 100 mM NaOAc | 11.6% | 5.2 | clear colloid |
| | | pH 4.4 | | | |
| DD | PDLLA-MePEG 15-5 | 6% sucrose 100 mM NaOAc | 11.6% | 5.2 | clear colloid |
| | | pH 4.4 | | | |
| DF | PDLLA-MePEG 30-5 | 6% HPβCD 100 mM NaOAc | 11.6% | 5.2 | cloudy, |
| | | pH 4.4 | | | translucent |
| DG | PDLLA-MePEG 15-5 | 100 mM NaOAc pH 6.0 | 11.6% | 5.2 | clear colloid |
| DH | PDLLA-MePEG 15-5 | 6% HPβCD 100 mM NaOAc | 11.6% | 5.2 | clear colloid |
| | | pH 6.0 | | | |
| DK | PDLLA-MePEG 15-5 | 100 mM NaOAc pH 7.9 | 11.6% | 5.2 | clear colloid |
| DO | PDLLA-MePEG 15-5 | 0.01% PS80 100 mM NaOAc | 10.1% | 5.0 | colloid |
| | | pH 7.9, then 6% HPβCD | | | |
| DP | PDLLA-MePEG 15-5 | 0.3% PS80 6% HPβCD | 10.2% | 5.1 | colloid |
| | | 100 mM NaOAc pH 7.9 | | | |
| DR | PDLLA-MePEG 15-5 | 0.3% PS80 100 mM NaOAc pH | 10.4% | 5.2 | colloid |
| | | 7.9, then 6% HPβCD | | | |
| DS | PDLLA-MePEG 15-5 | 0.2% F68 100 mM NaOAc pH | 10.6% | 5.4 | colloid |
| | | 7.9, then 6% HPβCD | | | |
| DT | PDLLA-MePEG 15-5 | 0.2% F68 100 mM NaOAc pH | 10.7% | 5.5 | colloid |
| | | 7.8, then 5% HPβCD | | | |
| DU | PDLLA-MePEG 15-5 | 100 mM NaOAc pH 7.9 | 10.4% | 5.5 | colloid |
| DV | PDLLA-MePEG 15-5 | 100 mM NaOAc pH 4.4 | 10.4% | 5.4 | colloid |
| DW | PDLLA-MePEG 15-5 | 100 mM NaOAc pH 7.9 | 10.5% | 5.2 | colloid |
| DX | PDLLA-MePEG 15-5 | 100 mM NaOAc pH 7.9, then | 10.7% | 5.4 | clear colloid |
| | | 0.1% PS80 and 10% HPβCD | | | |

### Freeze-Thaw Stable Cinacalcet Polymer Micelle Formulations

The polyesters in the diblock copolymers degrade through hydrolysis in presence of water. To increase stability, the formulations can be stored frozen. The freeze thaw stabilities of polymeric micellar cinacalcet formulations are evaluated (Table 8). Without protectant, the polymeric micelles aggregated upon freeze-thaw. Sucrose and trehalose provide some protect from aggregation. HPBCD provides the best protecting effect. No significant aggregation is observed.

TABLE 8
Freeze-Thaw Stability of Polymeric Micellar Cinacalcet Formulations

| | | | | API | | Appearance |
|---|---|---|---|---|---|---|
| | | | API | base | | (1:15 NTU) |
| Formu- | | | loading, | conc, | Appearance | after FT (3x |
| lation | Polymer | buffer | % | mg/ml | (1:15 NTU) | 12 hr-12 hr) |
| BC | PDLLA-MePEG | 100 mM NaOAc pH 6.0 | 10.2% | 4.6 | clear colloid | hazy colloid |
| | 11.7-5 | add 6% sucrose | | | | slightly hazy |
| | | add 6% trehalose | | | | slightly hazy |
| | | add 6% HPβCD | | | | colloid |
| BD | PDLLA-MePEG | 20 mM NaOAc pH 6.0 | 10.2% | 4.6 | colloid | hazy colloid |
| | 11.7-5 | add 6% sucrose | | | | slightly hazy |
| | | add 6% trehalose | | | | slightly hazy |
| | | add 6% HPβCD | | | | colloid |
| BE | PDLLA-MePEG | 20 mM HOAc | 10.2% | 4.4 | colloid | hazy colloid |
| | 11.7-5 | add 6% sucrose | | | | slightly hazy |
| | | add 6% HPβCD | | | | colloid |
| BF | PDLLA-MePEG | 20 mM NaOAc pH | 19.8% | 8.9 | clear colloid | colloid |
| | 11.7-5 | 6.0, 6% HPβCD | | | | |
| BJ | PDLLA-MePEG | 20 mM NaOAc pH | 19.8% | 8.9 | colloid | colloid |
| | 11.7-5 | 4.4, 6% HPβCD | | | | |
| BL | PDLLA-MePEG | 20 mM NaOAc pH | 11.2% | 5.1 | colloid | hazy colloid |
| | 11.7-5 | 6.0, 6% sucrose | | | | |
| BS | PDLLA-MePEG | 80 mM NaOAc pH | 11.2% | 5.1 | colloid | hazy colloid |
| | 20-5 | 6.0, 6% sucrose | | | | |
| BU | PDLLA-MePEG | 20 mM NaOAc pH | 11.2% | 5.1 | colloid (21.2) | colloid (22.5) |
| | 11.7-5 | 4.4, 3% HPβCD | | | | |
| BW | PDLLA-MePEG | 80 mM NaOAc pH | 11.2% | 5.1 | hazy colloid (51.3) | hazy colloid (49.7) |
| | 20-5 | 6.0, 6% HPβCD | | | | |
| BY | PDLLA-MePEG | 100 mM NaOAc pH | 20.2% | 10.3 | colloid (22.2) | colloid (24.3) |
| | 11.7-5 | 6.0, 6% HPβCD | | | | |
| CA | PDLLA-MePEG | 100 mM NaOAc pH | 20.2% | 10.3 | colloid (20.5) | slight aggregated (23.1) |
| | 11.7-5 | 4.4, 6% HPβCD | | | | |
| CB | PDLLA-MePEG | 100 mM NaOAc pH | 20.2% | 10.3 | hazy colloid (37.2) | hazy colloid (36.9) |
| | 20-5 | 4.4, 6% HPβCD | | | | |
| CC | PDLLA-MePEG | 100 mM NaOAc pH | 11.2% | 5.1 | colloid (21.3) | colloid (21.8) |
| | 11.7-5 | 6.0, 6% HPβCD | | | | |
| CD | PDLLA-MePEG | 100 mM NaOAc pH | 11.2% | 5.1 | hazy colloid (35.6) | hazy colloid (47.1) |
| | 20-5 | 6.0, 6% HPβCD | | | | |
| CE | PDLLA-MePEG | 100 mM NaOAc pH | 11.2% | 5.1 | colloid (19.1) | colloid (24.1) |
| | 11.7-5 | 4.4, 6% HPβCD | | | | |
| CF | PDLLA-MePEG | 100 mM NaOAc pH | 11.2% | 5.1 | hazy colloid (38.9) | hazy colloid (42.5) |
| | 20-5 | 4.4, 6% HPβCD | | | | |
| CG | PDLLA-MePEG | 100 mM NaOAc pH | 20.0% | 10.1 | colloid (36.8) | hazy clear (38.8) |
| | 11.7-5 | 4-6, 6% HPβCD | | | | |
| CH | PDLLA-MePEG | 100 mM NaOAc pH | 20.0% | 9.9 | clear colloid (9.7) | colloid (12.8) |
| | 15-5 | 4.4, 6% HPβCD | | | | |
| CI | PDLLA-MePEG | 100 mM NaOAc pH | 20.0% | 9.9 | clear colloid (6.7) | colloid (8.2) |
| | 11.7-5 | 4.4, 6% HPβCD | | | | |
| CJ | PDLLA-MePEG | 100 mM NaOAc pH | 11.1% | 5.0 | colloid (12.8) | colloid (14.1) |
| | 15-5 | 4.4, 6% HPβCD | | | | |
| CK | PDLLA-MePEG | 100 mM NaOAc pH | 11.1% | 5.0 | colloid (8.2) | colloid (20.3) |
| | 11.7-5 | 4.4, 6% HPβCD | | | | |
| CO | PDLLA-MePEG | 100 mM NaOAc pH 4.4 | 11.3% | 5.0 | clear colloid | NA |
| | 15-5 | add 6% HPβCD | | | | colloid |
| CP | PDLLA-MePEG | 100 mM NaOAc pH 4.4 | 12.0% | 5.4 | clear colloid | NA |
| | 11.7-5 | add 6% HPβCD | | | | colloid |

### Lyophilization Stable PDLLA-MePEG Cinacalcet Micelle Formulations

Lyophilization stable formulations, especially for water-insoluble diblock copolymers (Table 1), are not readily achievable but is desired for long term storage stability. Various excipients are evaluated as lyophilization protectants. Formulation DW (Example 6) is added with different excipients and lyophilized, followed by reconstitution. Table 9 shows that 10% HPβCD effectively protected cinacalcet loaded polymer from aggregation during lyophilization. A small amount of PS 80 surfactant further facilitated reconstitution.

TABLE 9
Evaluation of lyophilization stable formulations

| Formulation | Excipients | Cake appearance | Reconstitution appearance |
|---|---|---|---|
| DS | 10% HPβCD | White cake | All dissolved, clear colloid |
| DU | 10% HPβCD | White cake | All dissolved, clear colloid |
| DV | 10% HPβCD + 0.1% PS80 | White cake | All dissolved, clear colloid |
| DW | 10% HPβCD + 0.3% PS80 | White cake | All dissolved, clear colloid |
| DX | 10% HPβCD + 0.5% PS80 | White cake | All dissolved, clear colloid |

### Example 7 (Formulation DX)

0.6 g cinacalcet HCl, 5 g PDLLA-MePEG 15-5, and 60 mL acetone were combined to make an organic solution. The above solution was added to 100 mL of 0.1M NaOAc aqueous solution with pH 7.8 with magnetically stirring. Acetone and minor amount of water were evaporated, giving 85 g colloidal formulation. The resultant mixture was added with 10 g HPBCD, 1 mL of 10% PS80, and QS to 100 g (about 100 mL) with water. Clear colloidal polymeric micellar cinacalcet formulation was obtained. The pH was 5.2 and the 1:15 dilution turbidity is 10.7 NTU. 1 mL of the formulation was added to 3 mL vial each and lyophilized. The lyophilization cycle was freezing: 5° F./min ramping, −35° F. for 2 hrs, −5° F. for 2 hrs, −35° F. for 3 hrs; drying: −35° F. for 10 hrs, 25° F. for 8 hrs, then 75° F. for 8 hrs, with pressure less than 200 mTorr.

White cakes were obtained. Reconstitution with water or other aqueous solutions resulted in colloidal micelle solution (15× diluted turbidity=12 NTU).

### Physical Evaluation of Cinacalcet Polymer Micelle Formulations

The appearance, pH, turbidity of 15 times diluted solutions, and UV-Vis absorbance were used to test and screen the formulations. Release of cinacalcet from polymeric micellar formulations, into 37° C. pH 7.4 20 mM PBS buffer containing 0.1% PS80, through a 12-14 kD dialysis membrane, were measured (FIG. 4). Cincalcalcet concentration in the PBS buffer were determined by UV absorbance at 282 nm. The results showed sustained release of cinacalcet from the polymeric micellar formulations. These results indicate that cinacalcet associated with the diblock copolymer strongly, which may provide long-lasting circulation in blood.

### Unbound Fractions of Cinacalcet

The unbound cinacalcet fractions in plasma were determined. The cinacalcet buffer solution and cinacalcet polymer micelles were mixed with rabbit plasma at 1:4 ratio, incubated at 37° C., and filtered through 30 kD ultrafiltration membrane. The filtrate and unfiltered plasma were mixed with acetonitrile at a ratio of 1:4, and the cinacalcet concentrations were determined by an RP-HPLC. Table 10 presents unbound (filtered) and total (unfiltered) cinacalcet concentrations, and calculated % unbound fractions (unbound concentration divided by total concentration). As shown in Table 10, cinacalcet polymer micelle results in 20+ folds lower unbound fraction than cinacalcet buffer solution.

TABLE 10
Unbound and total cinacalcet concentrations- polymeric micellar formulations

| | Recon | Total | Unbound con- | | | |
|---|---|---|---|---|---|---|
| Formulation | conc., | conc., | centration, mg/mL | | % Unbound | |
| ID | mg/ml | mg/mL | 5 min^{c} | 15 min^{c} | 5 min^{c} | 15 min^{c} |
| cinacalcet^{a} | 1 | 0.84 ± 0.01 | 0.092 ± 0.016 | NT | 11.0% ± 1.9% | NT |
| NB009-030^{b} | 1 | 0.94 ± 0.04 | 0.004 ± 0.001 | NT | 0.41% ± 0.13% | NT |
| NB009-030^{b} | 10 | 8.7 | 0.012 | 0.041 | 0.14% | 0.47% |

^{a}cinacalcet dissolved in 100 mM NaOAc/HOAc pH 4.0, 1 mg/mL
^{b}lyophilized cinacalcet polymeric micelle reconstituted with water to cinacalcet conc 10 mg/mL, further diluted with D5W to 1 mg/mL
^{c}1:4 mixed with rabbit plasma and incubated at 37° C. for 5 or 15 min

### Evaluating Blood Compatibility of Cinacalcet Polymer Micelle Formulations

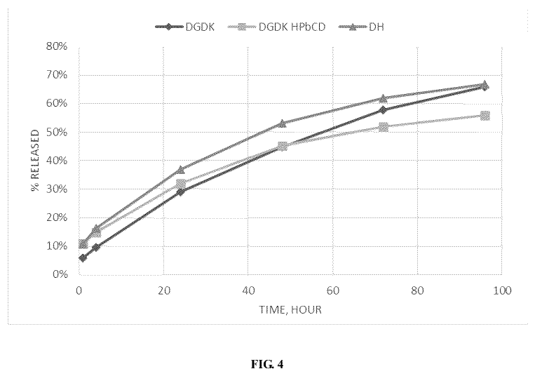

Fresh blood from a human donor was diluted 13.5 times with 0.9% saline. To 4.9 mL of the diluted whole blood, 0.1 mL cinacalcet formulations at 1 mg/mL were added. The glass vial containers were gently mixed for 0.5 hour. After settling of the blood cells, the color of the supernatant was observed (FIG. 5a). As shown, the free cinacalcet solution and micellar formulation at 30%-70% cinacalcet loading had reddish color at supernatant, with free cinacalcet the most reddish. The reddish color is from cell lysis, indicating blood incompatible. In contrary, polymeric micelles with 5%-10% cinacalcet loading had the same non-reddish color as the negative saline control, indicating that these formulations are compatible with blood cells. The polymeric micellar formulations effectively entrapped cinacalcet and increased cinacalcet's blood compatibility. Interestingly, with the same polymer and at the same drug loading and concentration, the utilization of acetate buffer pH 6.2 increased blood compatibility (formulation AL vs AN).

Another study compared cinacalcet buffer solution (100 mM NaOAc/HOAc pH 4.0) and cinacalcet polymer micelle formulations. A test tube was added with 2.5 mL of 2% rabbit red blood cell, 2.2 mL NS, and 0.3 mL cinacalcet samples. Normal saline (NS) and water were used as negative and positive controls, respectively. The test tubes were gently mixed and incubated at 37° C. The supernatants were separated by centrifugation and the absorbance at 540 nm (A) were measured. The % of hemolysis was calculated by:

$$Hemolysis ( \% ) = ( {A}_{sample} - {A}_{negative control} ) / ( {A}_{positive control} - {A}_{negative control} )$$

Results in Table 11 show that the polymer micelle formulations significantly reduced hemolysis, comparing to cinacacet buffer solution. FIG. 5b provides an exemplary photograph of the hemolysis test results.

TABLE 11
Hemolysis of cinacalcet- polymeric micellar
formulation vs simple solution

| | | | Cinacalcet | |
|---|---|---|---|---|
| | Incubation time | | concentration, | |
| Formulation | (37° C.) | | mg/mL | Hemolysis |
| Cinacalcet buffer solution | 15-30 | min | 0.2 | 80.6% |
| Cinacalcet buffer solution | | | 0.05 | 55.9% |
| NB009-010#27 | | | 0.2 | 1.1% |
| NB010-010#30 | | | 0.2 | 1.8% |
| NB010-010#31 | | | 0.2 | 1.0% |
| NB010-010#34 | | | 0.2 | 0.3% |
| NB010-010#35 | | | 0.2 | 0.4% |
| NB010-006#17 | 30 | min | 0.2 | −3.0% |
| NB010-006#18 | | | 0.2 | −0.8% |
| NB010-006#19 | | | 0.2 | 1.4% |
| NB010-006#20 | | | 0.2 | −2.2% |
| NB010-010#25 | | | 0.2 | 2.6% |
| NB010-010#26 | | | 0.2 | −1.6% |
| NB010-010#27 | | | 0.2 | −2.6% |
| NB010-010#28 | | | 0.2 | −1.4% |
| NB010-010#29 | | | 0.2 | −1.8% |
| NB010-010#30 | | | 0.2 | −2.4% |
| NB010-010#31 | | | 0.2 | −1.0% |
| NB010-010#32 | | | 0.2 | −0.8% |
| NB010-010#33 | | | 0.2 | 4.0% |
| NB010-010#34 | | | 0.2 | 3.4% |
| NB010-010#35 | | | 0.2 | 3.0% |
| NB010-020#36 | | | 0.2 | −1.8% |
| NB010-020#37 | | | 0.2 | −2.2% |
| NB010-020#38 | | | 0.2 | −2.8% |
| NB009-030 | 5 | min | 2 | 3.7% |

| | | |
|---|---|---|
| NB009-030 | 1 | 4.3% |

### Evaluating Stability of Cinacalcet Polymer Micelle Formulations

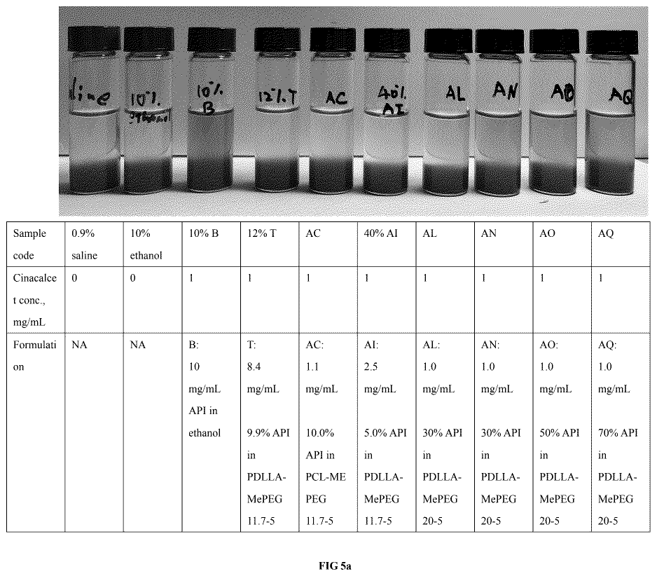

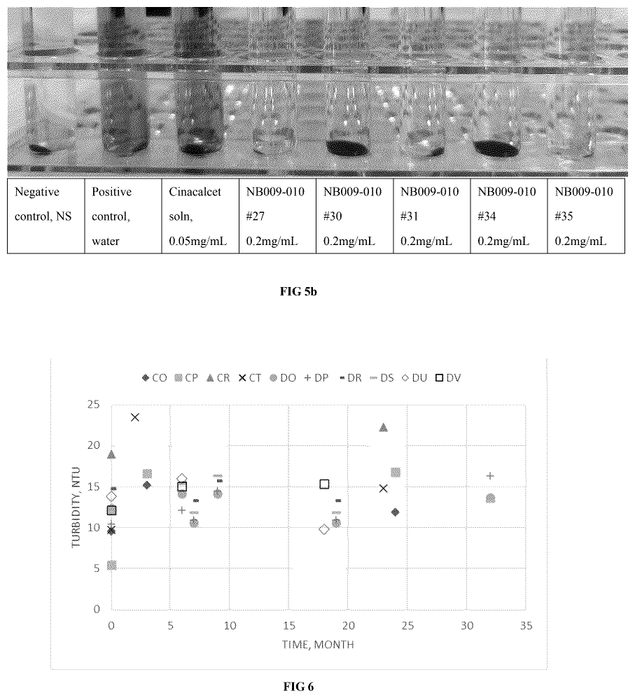

The polymeric micellar cinacalcet formulations, liquid or lyophilized, were stored at −20° C., 2-8° C., ambient, and 40° C. for stability observations. Physical appearance (before and after reconstitution for the case of lyophilized samples), turbidity (15× diluted NTU), pH, and uv absorbance were monitored at different storage time. At ambient and 40° C., both liquid and lyophilized samples showed significant changes in appearance (especially aggregation), turbidity, and pH (reduced pH due to hydrolysis of polyesters) in a short period. On the other hand, long-term storage stability was achieved at −20° C. for selected liquid formulations and at 2-8° C. for selected lyophilized formulations. No significant changes were observed in the tested parameters. FIG. 6 and FIG. 7 demonstrate that the turbidity, the most sensitive parameter, is stable over the storage period.

Additional storage stability data for lyophilized cinacalcet polymer micelles are provided in Table 12. Stability after reconstitution of the lyophilized and ethanol solutions formulations is exemplified in Table 13a and Table 13b, respectively.

TABLE 12
Stability of lyophilized cinacalcet polymeric micelle formulation (lot NB009-030) stored at 2-8° C.

| Attribute | 0 M | 3 M | 6 M |
|---|---|---|---|
| Appearance | White cake, some partial | white cake, no obvious | White cake, some partial |
| | shrinkage | defects | shrinkage |
| LOD | 4.09%-4.65% (n = 3) | 3.78% | 4.04% |
| Recon time, min | 5-90 (n = 6) | 19 | 28 |
| Recon appearance | Clear, colorless, practically | Clear, colorless, practically | Clear, colorless, practically |
| | free of particles | free of particles | free of particles |
| Recon turbidity, NTU | 0 h: 2.4-5.7 | 0 h: 4.3 | 0 h: 1.3 |
| | 6 h: 2.1-3.5 | 6 h: 3.6 | 6 h: 1.4 |
| | 24 h: 2.0-2.6 | 24 h: 3.6 | 24 h: 1.5 |
| | (n = 6) | | |
| Recon pH | 0 h: 5.17-5.58 | 0 h: 5.19 | 0 h: 5.26 |
| | 6 h: 5.15-5.41 | 6 h: 5.26 | 6 h: 5.28 |
| | 24 h: 5.13-5.33 | 24 h: 5.29 | 24 h: 5.17 |
| | (n = 6) | | |
| Cinacalcet content, mg/vial | 19.0 mg | 17.7 mg | 18.0 mg |
| Cinacalcet related substances | ND | ND | ND |
| Polymer MW and PDI | MW: 3418 | MW: 3383 | MW: 3189 |
| | PDI: 1.063 | PD: 1.047 | PD: 1.058 |
| PSD, average and PD | Avg: 12.77 nm; | Avg: 12.83 nm; | Avg: 12.57 nm; |
| | PD = 0.029 | PD = 0.052 | PD = 0.038 |

TABLE 13a
Stability of reconstituted lyophilized cinacalcet
polymeric micellar formulation (lot NB009-030)

| | 2-8° C. | | | 25° C. | | |
|---|---|---|---|---|---|---|
| | | Cinacalcet | | | Cinacalcet | |
| | | conc., | Related | | conc, | Related |
| Time, hr | Appearance | mg/mL^{a} | substances | Appearance | mg/mL^{a.} | substances |
| 0 | Clear solution | 8.6 | ND | Clear solution | 9.1 | ND |
| 6 | Clear solution | 9.1 | ND | Clear solution | 9.1 | ND |
| 24 | Clear solution | 8.9 | ND | Clear solution | 8.8 | ND |
| 48 | Clear solution | 8.7 | ND | Clear solution | 8.9 | ND |

ND = not detected
^{a}NB009-030 was added into 1.85 ml water to reconstitute, and stored at 4° C./25° C. respectively.

TABLE 13b
Stability of reconstituted cinacalcet ethanol solution
polymeric micellar formulation (ID SP-240708)^{a}

| Polymer used | PDLLA-PEG-PDLLA 1.1-3.35-1.1 | | PDLLA-PEG-PDLLA 1.3-6-1.3 | |
|---|---|---|---|---|
| Cinacalcet loading, % | 10 | 15 | 10 | 20 |
| Cinacalcet concentration, | 30 | 45 | 30 | 45 |
| mg/mL | | | | |
| Appearance - 0 hr | Colloidal | Colloidal | Colloidal | Colloidal |
| Appearance - 3 hr | Colloidal | Colloidal | Colloidal | Colloidal |
| Appearance - 24 hr | Colloidal | Colloidal | Colloidal | Colloidal |
| Appearance - 48 hr | Colloidal | Colloidal | Colloidal | Colloidal |

^{a}diluted 10x in water or D5W; samples stored at room temperature about 25° C.

### In Vivo Evaluation of Cinacalcet Polymer Micelle Formulation

The general safety, injection site reactions, pharmacokinetics, and efficacy (PTH reduction) of cinacalcet polymer micelle were evaluated in adult, healthy, and male sprague dowley (SD) rats in 4 separate studies, with various dose regime. Cinacalcet buffer solution was included for comparison. Normal saline and polymer micelle vehicles were also tested.

### Rat Study 1

Healthy, adult, male SD rats were divided into 4 groups, 6 rats per group and dosed with normal saline (NS), two cinacalcet polymer micelles (lot NB007-010A and NB007-013), and cinacalcet buffer solution (lot NB 007-017) at 3 mg/kg cinacalcet on day 1, 3, 5, 8 by bolus intravenous injection through tail vein. General health was observed and body weights were recorded. Blood samples were collected at 0, 1, 4, 8, 24 hours after each dosing by cutting tail tip. The PTH levels in plasma were measured by ELISA kits.

During the course of dosing, black tails were developed in all rats in the cinacalcet buffer solution group and in some rats in the cinacalcet polymer micelle group, due to drug irritation. No black tail occurred in the NS negative control group. As a result, some blood samples could not be collected from the rats with black tails. Table 14 provides a summary of number of rats with black tails. Table 15 presents the body weights, where the negative control group gained the most and the cinacalcet buffer solution group gained the least. The reduction of PTH levels is presented in FIG. 8. Similar reductions were observed in cinacalcet buffer solution and cinacalcet polymer micelle groups.

The results show that polymer micelles improve safety, reduce irritation and phlebitis (black tail), and is efficacious in reducing PTH levels.

TABLE 14
Number of adult male SD rats with blood being collected via cutting tail^{a} after
iv dosing of normal saline and 3 mg/kg cinacalcet. Bloods were not collected
due to development of black tail after cinacalcet dosing (Rat study 1).

| Group (n = 6) | negative control | LS-001^{b} | LS-002^{c} | LS-003^{d} |
|---|---|---|---|---|
| Formulation | Normal saline | Cinacalcet polymer | Cinacalcet polymer | Cinacalcet buffer |
| | | micelle (lot | micelle (lot | solution (lot |
| | | NB007-010A) | NB007-013 diluted) | NB007-017) |
| Cinacalcet conc, | 0 | 5 | 5 | 2 |
| mg/mL | | | | |
| Dosing volume, | 1.5 | 0.6 | 0.6 | 1.5 |
| mL/kg | | | | |
| Day 1 | 6/6 | 6/6 | 6/6 | 6/6 |
| Day 3 | 6/6 | 6/6 | 6/6 | 2/6 |
| Day 5 | 6/6 | 5/6 | 4/6 | 0/6 |
| Day 8 | 6/6 | 3/6 | 2/6 | 0/6 |

^{a}Blood collected at 0, 1, 4, 8, 24 hours after each dosing by cutting tail.
^{b}5 mg/mL cinacalcet, 45 mg/mL PDLLA-MePEG 15-5, 50 mg/mL HPßCD, 1 mg/mL PS80, in 100 mM NaOAc buffer (NB007-017).
^{c}5 mg/mL cinacalcet, 49 mg/mL PDLLA-MePEG 2-2, 5% v/v ethanol, 5% w/v sucrose, in 100 mM NaOAc buffer (NB007-017).
^{d}2 mg/mL cinacalcet, 4% v/v ethanol, in 100 mM HOAc/NaOAc pH 4.0 buffer (NB007-017).

TABLE 15
Body weight of adult male SD rats after iv dosing
of NS and 3 mg/kg cinacalcet (Rat study 1)

| Day | negative control | LS-001 | LS-002 | LS-003 |
|---|---|---|---|---|
| 1 | 311.0 ± 10.3 | 300.2 ± 12.0 | 301.3 ± 3.6 | 304.0 ± 8.5 |
| 3 | 324.2 ± 13.2 | 308.2 ± 13.7 | 313.0 ± 5.4 | 304.2 ± 12.2* |
| 5 | 338.3 ± 18.1 | 316.0 ± 16.2* | 326.7 ± 8.0 | 301.5 ± 13.3** |
| 8 | 362.7 ± 18.7 | 332.0 ± 17.8* | 343.2 ± 10.4* | 318.7 ± 13.3*** |

Comparing to the negative control group,
*P < 0.05,
**P < 0.01,
***P < 0.001

### Rat Study 2

Healthy, adult, male SD rats were divided into 6 groups, 4 rats per group and dosed with normal saline (NS), cinacalcet polymer micelles (cinacalcet doses: 2, 1, 0.5, 0.25 mg/kg; 10 mL/kg), and cinacalcet buffer solution (cinacalcet dose: 0.25 mg/kg; 10 mL/kg) on day 1, 3, 5, 8, 10, and 12 by bolus intravenous injection through tail vein. General health was observed and body weights were recorded. Blood samples were collected on days 1, 8, and 12 through eye socket. Sampling times on day 1 and day 12 were 0, 1, 2, 4, 8, and 24 hrs. Sampling times on day 8 were 0, 1, 24 hrs. Cinacalcet concentrations in plasma were measured by LC-MS.

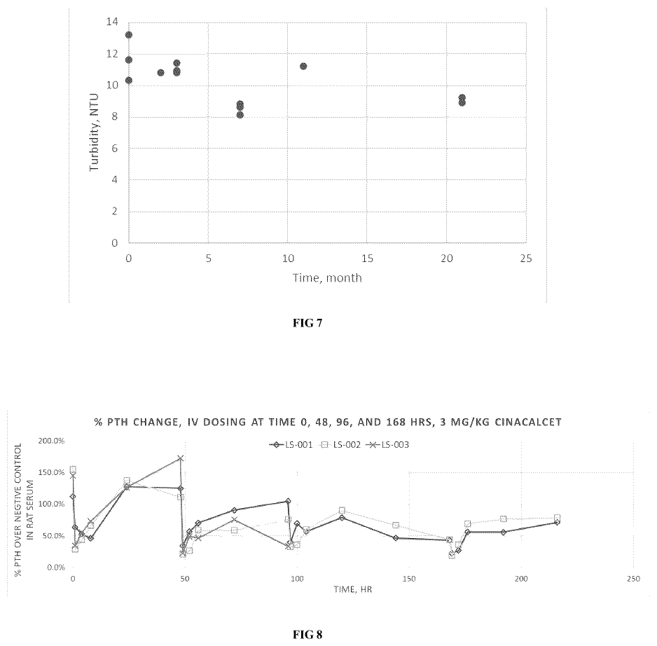

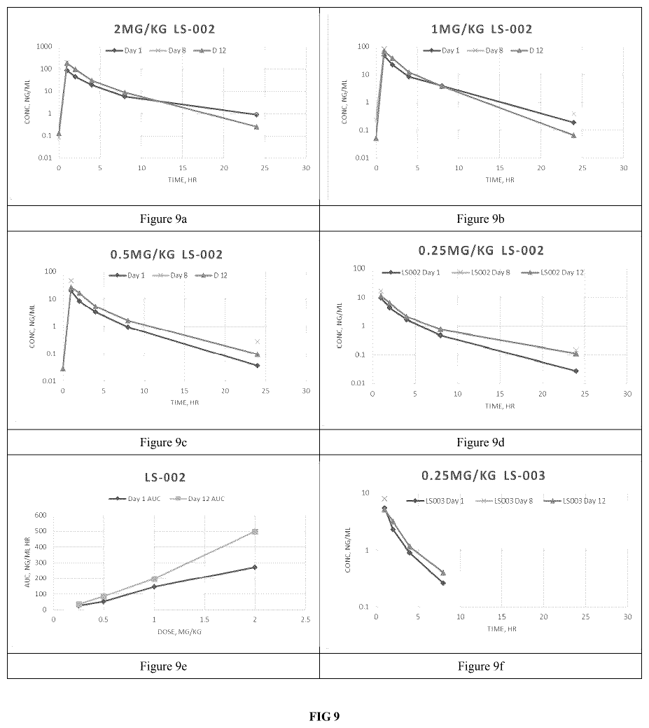

No black tail occurred in any of the groups. The rats appear generally healthy. Table 16 presents the body weights, where the negative control group and the 0.25 mg/kg cinacalcet polymer micelle groups gained the most weight. The rest of the groups gained less weight. The cinacalcet concentrations in plasma are presented in FIG. 9. The AUC of polymer micelle groups correlate linearly to the administered doses and shows cinacalcet accumulation from repeated injections.

The results show that polymer micelles are well tolerate. The pharmacokinetics profiles show linear dose responses and cinacalcet accumulations from repeated injections.

TABLE 16
Body weight after iv dosing of cinacalcet in adult male SD rats (4 per group)^{a} (Rat study 2)

| Group | Negative | | | | | | | | | | |
|---|---|---|---|---|---|---|---|---|---|---|---|
| (n = 4) | control | LS-002^{c} | | | | | | | | LS-003^{d} | |
| Formulation | NS | Cinacalcet polymer micelle | | | | | | | | Cinacalcet | |
| | | | | | | | | | | buffer solution | |
| Cinacalcet | 0 | 2 | | 1 | | 0.5 | | 0.25 | | 0.25 | |
| dose, mg/kg | | | | | | | | | | | |
| Injection | 10 | 10 | | 10 | | 10 | | 10 | | 10 | |
| volume, | | | | | | | | | | | |
| mL/kg | | | | | | | | | | | |
| Cinacalcet | 0 | 0.2 | | 0.1 | | 0.05 | | 0.025 | | 0.025 | |
| conc^{b}, mg/mL | | | | | | | | | | | |
| Day 1 | 259.0 ± 11.7 g | 258.0 ± 3.7 | g | 249.3 ± 9.6 | g | 254.8 ± 6.4 | g | 253.0 ± 5.5 | g | 249.5 ± 8.7 | g |
| Day 3 | 269.8 ± 16.6 g | 264.3 ± 3.9 | g | 260.3 ± 8.1 | g | 274.0 ± 4.2 | g | 269.3 ± 12.8 | g | 264.3 ± 7.5 | g |
| Day 5 | 286.8 ± 13.9 g | 279.8 ± 6.1 | g | 279.0 ± 5.6 | g | 284.0 ± 13.0 | g | 285.3 ± 9.5 | g | 284.0 ± 9.1 | g |
| Day 8 | 311.0 ± 18.9 g | 289.5 ± 26.7 | g | 305.8 ± 14.8 | g | 298.3 ± 35.0 | g | 317.5 ± 29.7 | g | 307.0 ± 24.9 | g |
| Day 10 | 306.5 ± 22.8 g | 289.3 ± 19.2 | g | 305.3 ± 34.9 | g | 306.3 ± 45.5 | g | 327.0 ± 52.0 | g | 318.8 ± 32.7 | g |
| Day 12 | 321.8 ± 29.9 g | 295.3 ± 24.8 | g | 303.8 ± 45.9 | g | 307.8 ± 40.5 | g | 338.5 ± 79.9 | g | 304.3 ± 45.0 | g |

| | |
|---|---|
| Comments | No black tails developed throughout the study. Rats showed normal healthy behaviors. |

^{a}bolus iv injection through tail vein on day 1, 3, 5, 8, 10, and 12; blood sampling on day 1, 8, and 12 through eye pocket; sampling time on day 1 and day 12: 0, 1, 2, 4, 8, 24 hrs; sampling time on day 8: 0, 1, 24 hrs; observed general health, tail color, and body weight; cinacalcet concentrations in plasma measured by LC-MS.
^{b}LS-002 and LS-003 were diluted with normal saline to respective cinacalcet concentration.
^{c}5 mg/mL cinacalcet, 49 mg/mL PDLLA-MePEG 2-2, 5% v/v ethanol, 5% w/v sucrose, in 100 mM NaOAc buffer (NB007-017).
^{d}2 mg/mL cinacalcet, 4% v/v ethanol, in 100 mM HOAc/NaOAc pH 4.0 buffer (NB007-017).

### Rat Study 3

Healthy, adult, male SD rats were divided into 3 groups, 6 rats per group and dosed with cinacalcet buffer solution (cinacalcet dose: 2 mg/kg, 2 mL/kg), cinacalcet polymer micelles (cinacalcet doses: 2 mg/kg, 2 mL/kg), and polymer vehicle (90 mg/kg, 2 mL/kg) on day 1, 3, 5, 8, 10, and 12 by bolus intravenous injection through tail vein. General health and tail appearance were observed and body weights were recorded. Blood samples were collected through eye socket. Right after the collection at 5 min and 1 hour, plasma was separated and filter through a 30 kD ultrafiltration microtube for measuring unbound cinacalcet. The cinacalcet concentrations were determined by LC-MS.

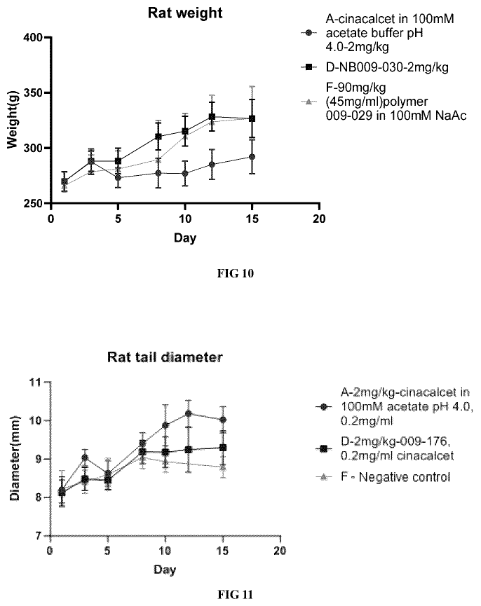

Rats in the cinacalcet buffer solution group struggled during the dose injections, while the other two group did not. The tail appearance was assessed using a scoring system presented in Table 17. Severe black tail and damages developed in the cinacalcet buffer group. Only minor reaction was observed in the cinacalcet polymer micelle group. No reaction was seen in the polymer vehicle group (Table 18). FIG. 10 provides the body weights, where the cinacalcet buffer group had the least weight gain. The cinacalcet concentrations in plasma, total vs unbound, are presented in Table 19. The unbound cinacalcet was below the lower quantitation limit, indicating cinacalcet extensively bounds to blood components. Similar total cinacalcet concentrations were observed between cinacalcet buffer solution and cinacalcet polymer micelle.

The results show that the cinacalcet polymer micelles significantly improved the safety over cinacalcet buffer solution by reducing injection site reactions, and are much more tolerated.

TABLE 17
Scoring of tail appearance

| Tail appearance | Scores |
|---|---|
| No erythema | 0 |
| Mild erythema (one quarter) | 1 |
| Moderate erythema (one third) | 2 |
| Severe erythema (one half) | 3 |
| Purple-red erythema to mild crusting | 4 |
| Mild dark purple (one quarter) | 5 |
| Moderate dark purple (one third) | 6 |
| Severe dark purple (half) | 7 |
| Dark purple to necrosis | 8 |
| Mild necrosis (one quarter) | 9 |
| Moderate necrosis (one third) | 10 |
| Severe necrosis (one half) | 11 |
| Ulceration (one quarter) | 12 |
| Ulceration (one third) | 13 |
| Ulceration (one half) | 14 |

TABLE 18
Scoring of tail reactions after repeated iv injections
of cinacalcet formulations (Rat study 3)

| Group (6 rats per group) | A | D | F |
|---|---|---|---|
| Formulation | Cinacalcet | Cinacalcet polymer | Polymer |
| | buffer | micelle (lot | vehicle^{c} |
| | solution^{a} | NB009-030)^{b} | |
| Dose, mg/kg | 2 | 2 | 0 |
| Injection volume, | 2 | 2 | 2 |
| mL/kg | | | |
| Cinacalcet conc., | 1 | 1 | 0 |
| mg/mL | | | |
| Day 1 | 0 | 0 | 0 |
| Day 3 | 8.33 (4-13) | 0.17 (0-1) | 0 |
| Day 5 | 9.57 (6-14) | 0 | 0 |
| Day 8 | 14 | 0 | 0 |
| Day 10 | 14 | 0 | 0 |
| Day 12 | 14 | 0.5 (0-3) | 0 |
| Comments | Rats struggled | normal | normal |
| | during dosing | | |
| | injections | | |

^{a}1 mg/mL cinacalcet in 100 mM acetate buffer pH 4.0
^{b}lyophilized cinacalcet polymer micelle lot NB009-030, reconstituted with water to cinacalcet 10 mg/mL, then diluted with D5W to concentrations
^{c}45 mg/mL polymer 009-029 dissolved in 100 mM NaOAc, 90 mg/kg dose
^{d}blood samples were collected though eye pocket at the following time points. Day 1: before dosing; Day 3: 0 (before dosing), 5 min, 1, 4, 8, 24, 48 hours; Day 5: 72 hours; Day 12: 0 (before dosing), 5 min, 1, 4, 8, 24, 48, 72 hours

TABLE 19
Unbound and total cinacalcet concentrations in rat plasma (Rat study 3)

| | Group A - cinacalcet buffer | | Group D - cinacalcet polymer micelle | |
|---|---|---|---|---|
| | Total | Unbound | Total | Unbound |
| | cinacalcet, | cinacalcet, | cinacalcet, | cinacalcet, |
| | ng/mL | ng/mL | ng/mL | ng/mL |
| Day 3 - 5 min | 270 ± 88 | <LLOQ | 188 ± 97 | <LLOQ |
| Day 3 - 1 hr | 82 ± 28 | <LLOQ | 85 ± 24 | <LLOQ |
| Day 12 - 5 min | 291 ± 184 | <LLOQ | 260 ± 310 | <LLOQ |
| Day 12 - 1 hr | 73 ± 37 | <LLOQ^{a} | 139 ± 57 | <LLOQ |

^{a}there was an outlier measured at 1.81 ng/mL. LLOQ (lower limit of quantitation) = 0.500 ng/mL.

### Rat Study 4

Healthy, adult, male SD rats were divided into 3 groups, 6 rats per group and dosed with cinacalcet buffer solution (cinacalcet dose: 2 mg/kg, 10 mL/kg), cinacalcet polymer micelle (cinacalcet dose: 2 mg/kg, 10 mL/kg), and 5% dextrose (D5W, 10 mL/kg) on day 1, 3, 5, 8, 10, and 12 by bolus intravenous injection through tail vein. General health and tail appearance were observed, and body weights and tail diameter were recorded.

Rats in the cinacalcet buffer solution group struggled during the dose injection, while the other two group did not. The tail appearance was assessed using a scoring system presented in Table 17. Severe black tail developed in the cinacalcet buffer group. Minor reaction was observed in the cinacalcet polymer micelle group. No reaction was seen in the D5W negative control group (Table 20). FIG. 11 presents the tail diameter close to the body, where the cinacalcet buffer solution group had the largest values. A larger diameter indicates inflammation or swelling due to the side reactions.

Again, the results show that the cinacalcet polymer micelles significantly improved the safety over cinacalcet buffer solution and are much better tolerated.

TABLE 20
Scoring of tail reactions after repeated iv injections
of cinacalcet formulations (Rat study 4)

| Group (6 rats per group) | A | D | F |
|---|---|---|---|
| Formulation | Cinacalcet | Cinacalcet | D5W |
| | buffer | polymer micelle | |
| | solution^{a} | (lot NB009-176)^{b} | control^{c} |
| Dose, mg/kg | 2 | 2 | 0 |
| Injection volume, | 10 | 10 | 10 |
| mL/kg | | | |
| Cinacalcet conc., | 0.2 | 0.2 | 0 |
| mg/mL | | | |
| Day 1 | 0 | 0 | 0 |
| Day 3 | 0 | 0 | 0 |
| Day 5 | 0 | 0 | 0 |
| Day 8 | 0 | 0 | 0 |
| Day 10 | 0.83 (0-2) | 0.3(0-1) | 0 |
| Day 12 | 8.83 (8-9) | 2.2(1-4) | 0 |
| Comments | Rats struggled | normal | normal |
| | during dosing | | |
| | injections | | |

^{a}1 mg/mL cinacalcet in 100 mM acetate buffer pH 4.0
^{b}lyophilized cinacalcet polymer micelle lot NB009-176, reconstituted with water to cinacalcet 10 mg/mL, then diluted with D5W to concentrations
^{c}5% dextrose water (D5W)

### INDUSTRIAL APPLICABILITY

The compositions of the present application are easy to manufacture, stable upon freezing and lyophilization, and stable in long term storage. The pharmaceutical compositions are blood compatible and reduce phlebitis and pain upon intravenous administration. The pharmaceutical formulations of the present application can be conveniently administered intravenously during dialysis resulting in increased patient compliance, increased bioavailability and reduced variabilities in pharmacokinetic parameters, eliminating “food effect” as compared to oral cinacalcet tablet formulations.

1. A pharmaceutical composition of cinacalcet, comprising micelles comprising cinacalcet, or a pharmaceutically acceptable salt thereof, and at least one amphiphilic block copolymer.

2. The pharmaceutical composition of claim 1, wherein the amphiphilic block copolymer is a diblock or triblock copolymer.

3. The pharmaceutical composition of claim 1, wherein the amphiphilic block copolymer is biodegradable polyester-block-methoxy polyethylene glycol (MePEG) or biodegradable polyester-block-polyethylene glycol (PEG)-block-polyester.

4. The pharmaceutical composition of claim 3, wherein the polyester block is selected from poly(D,L-lactide), poly(L-lactide), poly(D-lactide), polyglycolide, poly(ε-caprolactone), and copolymers or mixtures thereof.

5. (canceled)

6. The pharmaceutical composition of claim 4, wherein the molecular weight (Mₙ) of PDLLA is within the range from about 750 to about 50,000, and the molecular weights of MePEG and PEG are within the range from about 750 to about 20,000.

7-10. (canceled)

11. The pharmaceutical composition of claim 1, wherein the cinacalcet HCl loading in the amphiphilic block copolymer is from about 1% to about 70%.

12-13. (canceled)

14. The pharmaceutical composition of claim 1, further comprising one or more pharmaceutically acceptable excipients, carriers or diluents.

15-16. (canceled)

17. The pharmaceutical composition of claim 14, comprising α-CD, β-CD, γ-CD hydroxypropyl-β-cyclodextrin (HPBCD), and/or sulfobutylether-β-cyclodextrin (SBEβCD).

18. The pharmaceutical composition of claim 17, comprising HPBCD at a concentration within the range from about 3% to about 10% (wt/v %).

19. The pharmaceutical composition of claim 14, comprising a surfactant selected from polysorbates and poloxamers, wherein the polysorbates and poloxamers are selected from polysorbate 20, polysorbate 80, PLURONIC® 68.

20. (canceled)

21. The pharmaceutical composition of claim 19, comprising polysorbate 80 at a concentration within the range from about 0.01% to about 0.5% (wt/v %).

22. The pharmaceutical composition of claim 1, comprising a buffering agent, wherein the buffering agent comprises a mixture of acetic acid and sodium acetate or citric acid and sodium citrate, with a pH in the range from about 4.0 to about 7.9.

23. (canceled)

24. The pharmaceutical composition of claim 1, being an aqueous colloid.

25. The pharmaceutical composition of claim 24, being a clear to translucent colloid.

26. (canceled)

27. A unit dosage form comprising the pharmaceutical composition of claim 1.

28-29. (canceled)

30. A method for controlling parathyroid hormone (PTH) levels, comprising administering to a subject in need thereof a therapeutically effective amount of the pharmaceutical composition of claim 1.

31. A method for regulating calcium and phosphorus in blood, comprising administering to a subject in need thereof a therapeutically effective amount of the pharmaceutical composition of claim 1.

32. A method for treating primary hyperparathyroidism, comprising administering to a subject in need thereof a therapeutically effective amount of the pharmaceutical composition of claim 1.

33. A method for treating secondary hyperparathyroidism, comprising administering to a subject in need thereof a therapeutically effective amount of the pharmaceutical composition of claim 1.

34. A method for treating hypercalcemia, comprising administering to a subject in need thereof a therapeutically effective amount of the pharmaceutical composition of claim 1.

35-40. (canceled)

41. A method for preparing a composition of polymeric micellar cinacalcet, comprising:
dissolving at least one amphiphilic block copolymer and cinacalcet, or a pharmaceutically acceptable salt thereof, in an organic solvent, forming a first mixture; adding the first mixture to water or an aqueous solution with stirring; optionally removing the organic solvent, resulting in clear to translucent colloidal cinacalcet micelles; and optionally adding one or more pharmaceutically acceptable excipients, carriers or diluents;
or
dissolving at least one amphiphilic block copolymer and cinacalcet, or a pharmaceutically acceptable salt thereof, in an organic solvent, forming a first mixture; removing the organic solvent by evaporation, forming a matrix; adding water or an aqueous solution to the matrix, resulting in clear to translucent colloidal micelles; and optionally adding one or more pharmaceutically acceptable excipients, carriers or diluents.

42-43. (canceled)

44. The method of claim 41, wherein the amphiphilic block copolymer is a diblock or triblock copolymer.

45. The method of claim 44, wherein the diblock copolymer is biodegradable polyester-block-methoxy polyethylene glycol (MePEG), and the triblock copolymer is biodegradable polyester-block-polyethylene glycol (PEG)-block-polyester.

46. The method of claim 45, wherein the polyester block is selected from poly(D,L-lactide), poly(L-lactide), poly(D-lactide), polyglycolide, poly(ε-caprolactone), and copolymers and mixtures thereof.

47-48. (canceled)

49. The method of claim 46, wherein the molecular weight of PDLLA is within the range from about 750 to about 50,000, and the molecular weights of MePEG or PEG are within the range from about 750 to about 20,000.

50. (canceled)

51. The method of claim 45, further comprising storing the frozen polymeric micellar cinacalcet at about −20° C. for long term storage, or storing the lyophilized polymeric micellar cinacalcet at about 2° C. to about 8° C. for long term storage, or reconstituting the lyophilized polymeric micellar cinacalcet prior to administration.

52-64. (canceled)
